(19) United States
(12) Patent Application Publication
(10) Pub. No.: US 2006/0084631 A1
(43) Pub. Date: Apr. 20, 2006

(54) Antimicrobial charged polymers that exhibit resistance to lysosomal degradation during kidney filtration and renal passage, compositions and method of use thereof

(75) Inventor: Wayne D. Comper, New York, NY (US)

Correspondence Address: JONES DAY, 222 East 41st Street, New York, NY, 10017, US

(73) Assignee: Monash University

(21) Appl. No.: 11/291,226

(22) Filed: Dec. 1, 2005

Related U.S. Application Data
(63) Continuation of application No. 10/321,756, filed on Dec. 17, 2002, now abandoned.
(60) Provisional application No. 60/346,629, filed on Jan. 10, 2002.
(60) Provisional application No. 60/366,532, filed on Mar. 25, 2002.
(60) Provisional application No. 60/366,533, filed on Mar. 25, 2002.
(60) Provisional application No. 60/402,695, filed on Aug. 13, 2002.

(51) Int. Cl. A61K 31/737; A61K 31/721; A61K 31/715

(52) U.S. Cl. 514/54; 514/59

(57) ABSTRACT

Methods and compositions for treating or preventing microbial infection in mammals with sulfated polysaccharides wherein the polysaccharides have a degree of sulfation effective to enable maximal interaction of constituent sulfate groups with the microbe which causes the infection and wherein the sulfated polysaccharide is not substantially endocytosed or degraded by cell receptor binding in the mammal and thereby retains antimicrobial activity in vivo.

This application claims priority to U.S. Provisional Patent Application No. 60/346,692 filed Jan. 10, 2002; U.S. Provisional Patent Application No. 60/366,532 filed Mar. 25, 2002; U.S. Provisional Patent Application No. 60/366,533 filed Mar. 25, 2002; and U.S. Provisional Patent Application No. 60/402,695 filed Aug. 13, 2002, each of which is incorporated herein in its entirety by reference.

### 1. FIELD OF THE INVENTION

This invention relates to methods for treating or preventing microbial infections in mammals using “sulfated polysaccharides”. More particularly, this invention relates to methods of introducing a therapeutically effective amount of a charged and flexible sulfated polysaccharide having a certain percent sulfation range into the blood stream, lymphatic system and/or extracellular spaces of a human patient for the treatment, prevention or management of microbial infections. In particular, wherein the range is effective to enable maximal interaction of the sulfate groups with the microbe which causes the infection, and wherein the sulfated polysaccharide is not substantially endocytosed or degraded by cell receptor binding in the mammal, and thereby retains antimicrobial activity in vivo.

### 2. BACKGROUND OF THE INVENTION

Charged polysaccharides, particularly sulfated polysaccharides, have demonstrated potent antimicrobial activities in vitro. (Baba et al., Antiviral Res. 9:335-343, 1988; Ito et al., Antiviral Res. 7(36): 1-367, 1987). For example, sulfated polysaccharides such as dextran sulfate, heparin, and pentosan polysulfate have been reported to be potent inhibitors of HIV, paramyxoviruses, cytomegaloviruses, influenza viruses, semlikiviruses (Lüscher-Mattli et al., Arch Virol 130:317-326, 1993) and herpes simplex viruses in vitro (Baba et al., Antimicrob. Agents Chemotherapy 32:1742-45, 1988; Pancheva, Antiviral Chem Chemotherapy 4:189-191, 1993). However, these known compounds have disappointingly poor activity in vivo.

Dextran sulfate and heparin were first reported to inhibit HIV replication in vitro by Ito et al., Antiviral Res. 7:36 1 -367. 1987, Deringer et al. (U.S. Pat. No. 5,153,181) and Ueno and Kuno, Lancet 2:796-97, 1987. Later, several other sulfated polysaccharides were shown to inhibit HIV replication at concentrations believed to be below their respective cytotoxicity thresholds, e.g., pentosan sulfate (Baba et al., Antiviral Res 9: 335-343, 1988; Biesert et al., Aids 2(6):449-57, 1988), fuciodan (Baba et al., Antiviral Res 9:335-343, 1988), lambda-, kappa- and iota-carrageenan (Baba et al., Antiviral Res 9: 335-343, 1988), lentinan sulfate (Yoshida et al., Biochem. Pharmacol. 37(15):2887-91, 1988), mannan sulfate (Ito et al., Eur. J. Clin. Microbiol. Infect. Dis. 8: 191-193, 1989), dextrin sulfate (Ito et al. Antiviral Chem. Chemother., 2:41-44, 1991), sulfoevernan (Weiler et al., J Gen Virol 71:1957-1963, 1990), and sulfated cyclodextrins (Schols et al., J Acquired Immune Def. Syndr 4:677-85,1991.). However, these compounds have all proven ineffective in vivo, and at high concentrations cause thromobocytopenia, central nervous system side effects, hair loss, gastro-intestinal pain, anti-coagulation, and the like (Flexner et al., Antimicrob Agents Chemotherapy 35:2544-2550, 1991; Abrams et al., Annals of Internal Medicine (1989) 110:183-188; Hiebert et al., J. Lab & Clin. Med. 133:161-170 (1999)).

Certain sulfated polysaccharide compounds have also demonstrated anti-bacterial activity (Dalton et al., Bur J Biochem 195:179-184, 1991; Zarcha et al., Current Microbiol. 34:6-11, 1997; Pancake et al., J Cell Biol 117:1251-1257, 1992; Clark et al., Glyco J 14:473-9,1997), anti-chlamydial activity (Herold et al., Antimicrobial Agents and Chemotherapy 41:2776-2780, 1997, and Su and Caldwel, Infection and Immunity 66:1258, 1991) and anti-parasitic activity. Again, anti-microbial activity and anti-parasitic activity were observed in vitro, but the compounds proved ineffective in vivo (Dalton et al., Eur J Biochem 195:179-184, 1991; Pancake et al., J Cell Biol 117:1251-1257, 1992; Clark et al., Glyco J 14:473-9,1997).

Conventional or commercial dextran sulfate has a percent of sulfation of about 17-22%. It is widely accepted that increasing sulfur content increases activity of this material. For example, increasing sulfur content increases anti-coagulant activity. (Hirata et al., Biosci. Biotech. Biochem. 58(2):406-407, 1994). Similarly, it is widely accepted that increasing the sulfur content of sulfated polysaccharides increases their in vitro antiviral activity. See, e.g., Witvrouw et al., General Pharmacology 29 (4): 497-512, 1997; Nakashima et al., Jpn. J. Cancer Res. (Gann) 78:1164-68, 1987; and Baba et al., J. AIDS 493-499, 1990. Again, these studies have demonstrated a marked increase in the in vitro activity of sulfated polysaccharides with the increase in sulfation, although the lack of in vivo efficacy remains. Indeed, lack of in vivo efficacy and the in vivo toxicity of compounds with a high degree of sulfation has been an unsolvable problem to date.

Although there have been a limited number of studies of sulfated polysaccharides with lower percents of sulfation for specific uses, these materials have not been characterized with respect to both their molecular weight and their percent of sulfation. Significantly, these materials have been reported to be less active against retroviruses than polysaccharides with 17-22% sulfation. Id. Further, poorly characterized (if characterized at all), low molecular weight preparations have been studied in animals for activity against herpes virus as in EP Application 0 066 379 A2 with limited success. Pancheva S N. Antiviral Chem Chemotherapy 4:189-191, 1993.

One of the major reasons that dextran sulfate may not be active in vivo is that the material is not stable. Some indication of this has been published previously. Tritium labeled dextran sulfate mw 8,000 appeared to be depolymerised while in the blood circulation of rats over a 6-24 h period (Hartman N R, Johns D G, Mitsuya H. AIDS Res Hum Retroviruses 6: 805-811, 1990). Iodinated heparin and pentosan polysulphate are rapidly cleared from the circulation in man and returned in a desulfated form (MacGregor I R, Dawes J, Paton L, Pepper D S, Prowse C V, Smith M., Thromb Haem 51:321-325, 1984).

Considerable effort has been focused on improving the in vivo anti-viral activity of dextran sulfate by increasing its sulfation or modifying the use of conventional material. In one study, given the reported poor absorption of oral dextran sulfate, dextran sulfate was administered to a maximally tolerated dose by continuous infusion to subjects with symptomatic HIV infection for up to 14 days. (Flexner et al., Antimicrob Agents Chemotherapy 35:2544-2550, 1991). Continuous intravenous infusion of dextran sulfate was found to be toxic. The authors concluded that as a result of its toxicity and lack of any demonstration of beneficial effect in vivo, dextran sulfate is unlikely to have a beneficial effect in the treatment of HIV. Id. Indeed, the authors cautioned: “further clinical development of parenteral dextran sulfate as therapy for symptomatic HIV infection is not warranted and could prove to be hazardous. On the basis of the results of this study, caution is advised in the clinical evaluation of other polysulfated polyanions.” (Id. at 2549).

In a major study of the processing of dextran sulfate by glomerular endothelial cells, Applicant discovered that dextran sulfate binds to a cell surface receptor that would normally recognize highly sulfated polysaccharides such as heparin—like polysaccharides. On binding the dextran sulfate is endocytosed, desulfated but not depolymerised by lysosome sulfateases and exocytosed as desulfated dextran sulfate (Vyas et al. 1996). It was found that the uptake and endocytosis of dextran sulfate by the cell was critically dependent on the sulphur content or degree of sulfate substitution per glucose residue. Above 13% sulphur uptake by glomerular endothelial cells was significant whereas below 13% sulphur uptake and endocytosis was minimal. This means that charged polysaccharides with a particular critical sulphur content or critical sulfate substitution charge density along the polysaccharide chain may be processed differently by cells to which the circulation is exposed. Any organ in the body, particularly in the lymphatics where HIV production predominates, that mimics this process of cell receptor recognition, endocytosis and degradation would render the dextran sulfate inactive as an anti-viral drug in vivo. Highly sulfate materials, such as commercial dextran sulfate with 17-20% sulphur, may be rapidly taken up by cells, desulfated and tendered inactive in terms of antiviral activity whereas lower sulfated materials may not be taken up by cells and retain their antiviral activity.

In sum, although commercial dextran sulfate has been previously used in Japan for anticoagulation and hyperlipidemia, it has demonstrated poor activity against HIV in vivo or, dextran sulfate has been reported to have significant toxicity in mammals and HIV patients. (Mathis et al., Antimicrobial Agents & Chemotherapy 2147-2150, 1991; Flexner et al., Id. 2544-2550; Abrams et al., Annals of Internal Medicine 110:183-188 (1989); Hiebert et al., J. Lab & Clin. Med. 133:161-170 (1999)). Thus, there remains a need for a method for the in vivo activation of dextran sulfate against viral infection.

While the broad spectrum of in vitro activity made sulfated polysaccharides attractive as anti-microbial drug candidates in the past, there remains a need for a sulfated polysaccharide that is effective in vivo for the treatment or prevention of viral infections, bacterial infections and parasitic infections.

### 3. SUMMARY OF THE INVENTION

The inventor has discovered that lowering and controlling the degree of sulfation of flexible polysaccharides, and optionally controlling the molecular weight, yields a composition having both in vitro and in vivo antimicrobial activity. Such compositions can be used in methods to treat, prevent or manage microbial infections while reducing or avoiding adverse effects, e.g., toxicities associated with the oral or parenteral administration of conventional sulfated polysaccharides. More specifically, the inventor has discovered that preparations of sulfated α-1,6-polysaccharides having a controlled range of sulfation, e.g., with % sulfur above 6% and below 13%, are active in vivo against microbial infections.

Thus, the invention encompasses novel methods of treatment and novel pharmaceutical compositions which utilize such sulfated polysaccharides having a low percent of sulfation as compared to conventional dextran sulfate. For example, the invention encompasses sulfated polysaccharides having a percent of sulfur with respect to the simple sugar residue of greater than 6% and less than 13%, preferably greater than about 7% and less than 13%, more preferably greater than about 9% and less than 13%, most preferably 6%, 7%, 8%, 9%, 9.5%, 10%, 10.5%, 11%, 11.5%, 12%, 12.2%, 12.5% 12.8% or 12.9%. The sulfated polysaccharides are preferably sulfated dextrans having an α-1,6-glycosidic linkage.

The invention further encompasses sulfated polysaccharides having a molecular weight between 500 and 1,000,000, preferably above 5,000; more preferably above 25,000; most preferably above 40,000 particularly for oral or parenteral administration. Ranges of 5,000 to 1,000,000, 25,000 to 500,000 and 40,000 to 300,000 are also encompassed by the invention. However, for topical administration, the sulfated polysaccharide may have a molecular weight higher than 500,000 in a preferred embodiment. In an alternative embodiment, the composition has only about 10% variability in the molecular weight and preferably about 5% variation.

In a preferred embodiment of the invention, the sulfated polysaccharide is not cellulose sulfate, dextrin sulfate or cyclodextrin, but instead is an α-1,6-sulfated polysaccharide such as a sulfated dextran having a controlled range of sulfation, and, optionally, a specific molecular weight range. In an alternative embodiment, the sulfated polysaccharide is homogenous with respect to molecular weight, percent of sulfation or both.

In one aspect of the invention there is provided a method for introducing a therapeutically effective amount of a sulfated polysaccharide or salt thereof into the blood stream, lymphatic system and/or extracellular spaces tissue of a mammal comprising administering to the mammal at least one sulfated polysaccharide or a pharmaceutically acceptable salt or hydrate thereof having antimicrobial activity in vitro and having a percent of sulfation sufficient for retention of the anti-microbial activity in vivo. Preferably, the range of sulfation of the polysaccharide is effective to enable maximal interaction of constituent sulfate groups with the microbe which causes the infection, and wherein the sulfated polysaccharide is not substantially endocytosed or degraded by cell receptor binding in the mammal, and thereby retains antimicrobial activity in vivo.

In another aspect of the invention there is provided a method for treating or preventing a microbial infection comprising administering to a patient a therapeutically effective amount of sulfated dextran having a percent of sulfur greater than 6% and below 13%. In a preferred embodiment, sulfated dextran has a percent sulfation of above 6% or about or above: 6.5%, 7%, 7.5%, 8%, 8.5%, 9%, 9.5%, 10%, 10.5%, 11%, 11.5%, 12%, 12.2%, 12.5%, 12.8% or less than 13%. In a preferred embodiment the method is for treating or preventing a viral infection, including but not limited to DNA viruses and RNA viruses, particularly enveloped viruses whether DNA or RNA viruses. In a separate and preferred method the viruses to be treated include but are not limited to double-stranded DNA viruses, DNA reverse transcripting viruses, RNA reverse transcripting viruses, double-stranded RNA viruses, negative-sense single stranded RNA viruses, and positive-sense single-stranded RNA viruses.

In yet another aspect of the invention, there is provided a method for synthesizing a polysaccharide, or decreasing or increasing the degree of sulfation such that the sulfated polysaccharides are suitable for administration in vivo and are efficacious in vivo against viral infection. The method comprises providing the sulfated polysaccharides with a percent of sulfation sufficient to eliminate or reduce binding and internalization of the sulfated polysaccharides by high charge density polyanion cell receptors, or otherwise inactivate these compounds in vivo but sufficient to provide antimicrobial activity; and administering the sulfated polysaccharide to a mammal. In other words, the invention encompasses modifying the sulfation of a naturally occurring or commercially available sulfated polysaccharide to a range of sulfation effective to enable maximal interaction with the microbe and wherein the sulfated polysaccharide is not substantially endocytosed or degraded by cell receptor binding.

Separate aspects of the invention the invention encompass pharmaceutical compositions suitable for parenteral administration to a patient comprising a therapeutically or pharmaceutically acceptable amount of a polysaccharide of the invention; pharmaceutical compositions suitable for oral administration to a patient comprising a therapeutically or pharmaceutically acceptable amount of a polysaccharide of the invention; and pharmaceutical compositions suitable for topical administration to a patient comprising a therapeutically or pharmaceutically acceptable amount of a polysaccharide of the invention having a molecular weight greater than 500,000.

The microbial infections encompassed by the methods of the invention, particularly the specific viruses to be treated and specific sulfated dextrans to be used, are described in detail below.

### 3.1. DEFINITIONS

As used herein, the term “patient” means an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In certain embodiments, the patient is an infant, child, adolescent or adult. In addition, the patient includes immunocompromised patients such as HIV positive patients, cancer patients, and patients undergoing immunotherapy.

As used herein, a “therapeutically effective amount” refers to an amount of the compound of the invention or other active ingredient sufficient to provide a benefit in the treatment or management of the disease, to delay or minimize symptoms associated with the disease, or to cure or ameliorate the disease or infection or cause thereof. In particular, a therapeutically effective amount means an amount sufficient to provide a therapeutic benefit in vivo. Further, a therapeutically effective amount means an amount of a compound of the invention alone, or in combination with other therapies, that provides a benefit in the treatment or management of the disease, to delay or minimize symptoms associated with the disease, or to cure or ameliorate the disease or infection or cause thereof. Additionally, a therapeutically effective means an amount of therapeutic agent that provides a benefit in the treatment or management of the disease without being toxic to the patient. Used in connection with an amount of a compound of the invention, the term encompasses an amount that improves overall therapy, reduces or avoids symptoms or causes of disease, or enhances the therapeutic efficacy of or synergies with another therapeutic agent.

As used herein, a “prophylactically effective amount” refers to an amount of a compound of the invention or other active ingredient sufficient to result in the prevention of recurrence or spread of the disease. A prophylactically effective amount may refer to an amount sufficient to prevent initial disease or the recurrence or spread of the disease or the occurrence of the disease in a patient, including but not limited to those predisposed to the disease. In particular, a prophylactically effective amount with respect to a compound of the invention means an amount sufficient to result in the prevention of recurrence or spread of the disease in vivo. A prophylactically effective amount may also refer to an amount that provides a benefit in the prevention of the disease without being toxic to the patient. Further, a prophylactically effective amount with respect to a compound of the invention means an amount alone, or in combination with other agents, that provides a prophylactic benefit in the prevention of the disease. Used in connection with an amount of a compound of the invention, the term encompasses an amount that improves overall prophylaxis or enhances the prophylactic efficacy of or synergies with another prophylactic or therapeutic agent.

As used herein, “in combination” refers to the use of more than one prophylactic and/or therapeutic agents simultaneously or sequentially and in a manner that their respective effects are additive or synergistic.

As used herein, the terms “manage”, “managing” and “management” refer to slowing or preventing the progression or worsening of the disease but not curing the disease.

As used herein, the terms “prevent”, “preventing” and “prevention” refer to the prevention of the onset, recurrence, or spread of the disease in a subject resulting from the administration of an active ingredient before the disease or infection occurs.

As used herein, the terms “treat”, “treating” and “treatment” refer to the eradication or amelioration of the disease or infection itself, causes of the disease or symptoms associated with the disease. In certain embodiments, such terms refer to minimizing the spread or worsening of the disease or infection resulting from the administration of one or more prophylactic or therapeutic agents to a subject with such a disease.

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compound of the present invention include, but are not limited to, metallic salts made from aluminum, calcium, lithium, magnesium, potassium, sodium and zinc or organic salts made from lysine, NN′-dibenzylethylenediamine, chloroprocaine, choline, diethanolamine, ethylenediamine, meglumine (N-methylglucamine) and procaine.

As used herein and unless otherwise indicated, the term “optically pure” or “stereomerically pure” means a composition that comprises one stereoisomer of a compound and is substantially free of other stereoisomers of that compound. For example, a stereomerically pure a compound having one chiral center will be substantially free of the opposite enantiomer of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. Since the compounds of the invention are polysaccharides made of saccharides which can exist in either the D or L forms, the invention encompasses either or both D and L sugars. As such, for example, a stereomerically pure D sugar will be substantially free of the L form. In an alternative embodiment, the use of L forms of sulfated dextrans permits the use of a broader controlled range of sulfation from above 6% to about 20%. Thus, the methods and compositions disclosed herein include in an alternative embodiment the use of such levorotatory sugars or polymers made therefrom.

As used herein, the term “dextran” means a polysaccharide containing a backbone of D-glucose units linked predominantly α-D(1,6), composed exclusively of α-D-glucopyranosyl units differing only in degree of branching and chain length.

As used herein, the term “dextran sulfate sodium” or “dextran sulfate”, “conventional dextran sulfate”, or “commercial dextran sulfate” unless otherwise qualified means a α1,6-polyglucose containing approximately 17% sulfur with up to three sulfate groups per glucose molecule of varying molecular weight ranges, e.g., 4,000-500,000 Da.

As used herein, the terms “percent sulfation”, “percent of sulfation”, “percent of sulfate substitution” or “sulfation” means the percent of sulfur by molecular weight with respect to each simple sugar residue within the polysaccharide in question optionally including a counterion, e.g., molecular weight of sulfation in the composition/total weight. The percent of sulfation can be determined by elemental analysis of material which has been dialyzed to remove free sulfur, preferably of moisture/volatile free material dried in vacuo at 60° C. to a constant weight. Other methods of determining percent of sulfation are via moisture content analysis and titration. Sulfation is to be distinguished from “degree of substitution” or “equivalents” which is a measure of the number of sulfate groups per sugar moiety. However, it will be recognized by one of skill in the art that percent sulfation can be converted to a degree of substitution or equivalents and vice versa.

As used herein, the term “co-charged dextran polyanions is dextran substituted to varying degrees with any combination of carboxymethyl groups, sulfate groups and sulfonate groups.

As used herein, the term “periodate treated anionic polysaccharides” means any anionic polysaccharide that has been treated with periodate to open the sugar ring without depolymerization or to otherwise increase the flexibility of the polysaccharide in order to increase interaction with the microbe.

As used herein, the term “antimicrobial” includes antiviral; antibacterial, such as, for example, antichlamydial; antiparasitic, such as anti-Plasmodium or anti-fungal.

### 4. BRIEF DESCRIPTION OF THE DRAWINGS

FIG. 1 is a graph showing the amount of desulfation of circulating plasma commercial dextran sulfate as a function of time in plasma (n=3) in Sprague-Dawley rats. Values up to 24 hours were based on dextran sulfate existing in plasma after a bolus intravenous injection at zero time. The mean value at 168 hours was obtained from steady state osmotic pumps implanted subcutaneously in Sprague-Dawley rats.

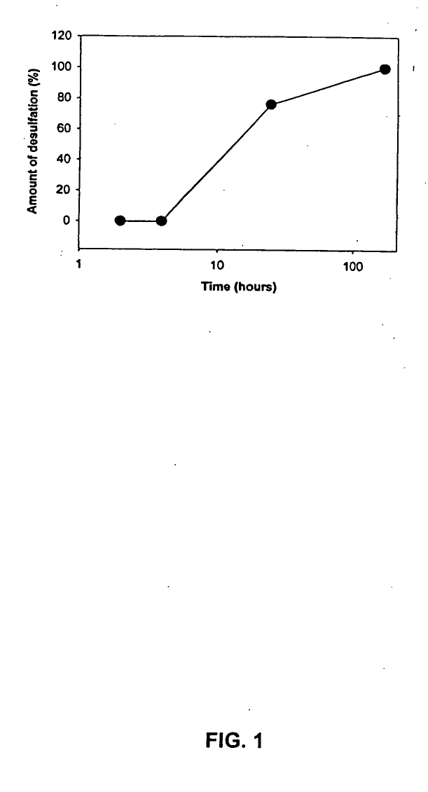

FIG. 2 is a graph showing the effective antiviral active concentration of polysaccharide material versus time after bolus iv injection (172 mg/kg) at time zero. Group 1: commercial dextran sulfate, mw-40,000 (n=1-3); Group 2: commercial dextran sulfate, mw-500,000 (n=3); Group 3: sulfated dextran 12.6% (DES 6 40 k) (N=4-6); group 4: sulfated dextran 12.2% (DES 6 500 k) (N=6): group 5: daily injections of DES 6 40 k for 6 days at 172/kg/day (n=4).

FIG. 3 is a schematic flowchart describing the preparation of sulfated dextrans of a specific percent of sulfation and molecular weights.

FIG. 4 is a profile of 40,000 mw tritium labeled sulfated dextran chromatography profile from ion exchange chromatography eluted from cation exchange resin with a linear sodium chloride gradient showing a high degree of homogeneity of degree of sulfate substitution.

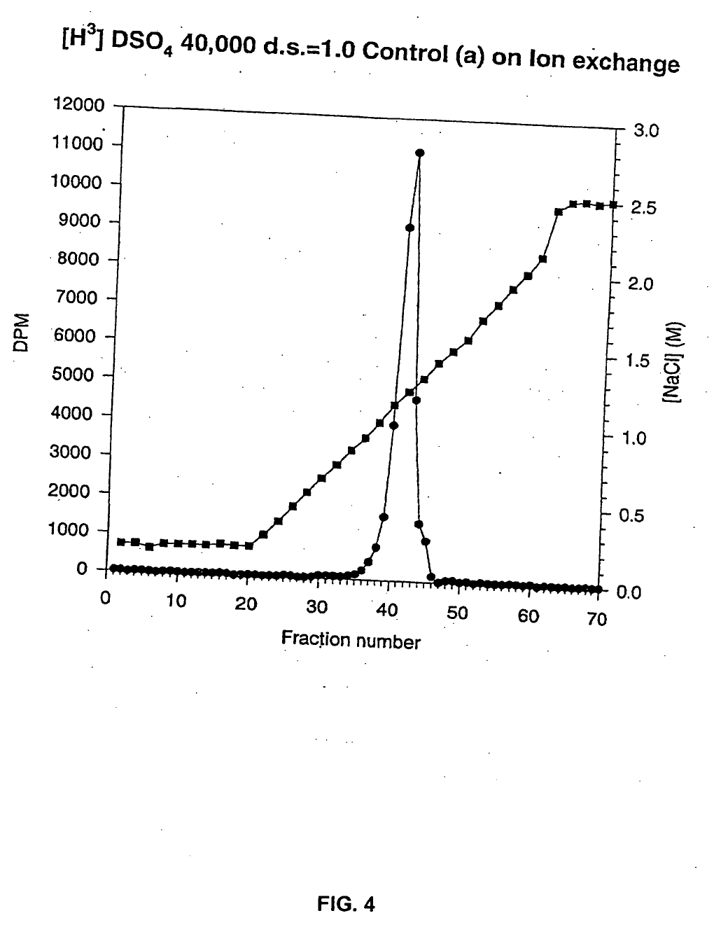

FIG. 5 is a profile of 500,000 mw tritium labeled sulfated dextran chromatography profile from ion exchange chromatography eluted from cation exchange resin with a linear sodium chloride gradient showing a high degree of homogeneity of degree of sulfate substitution.

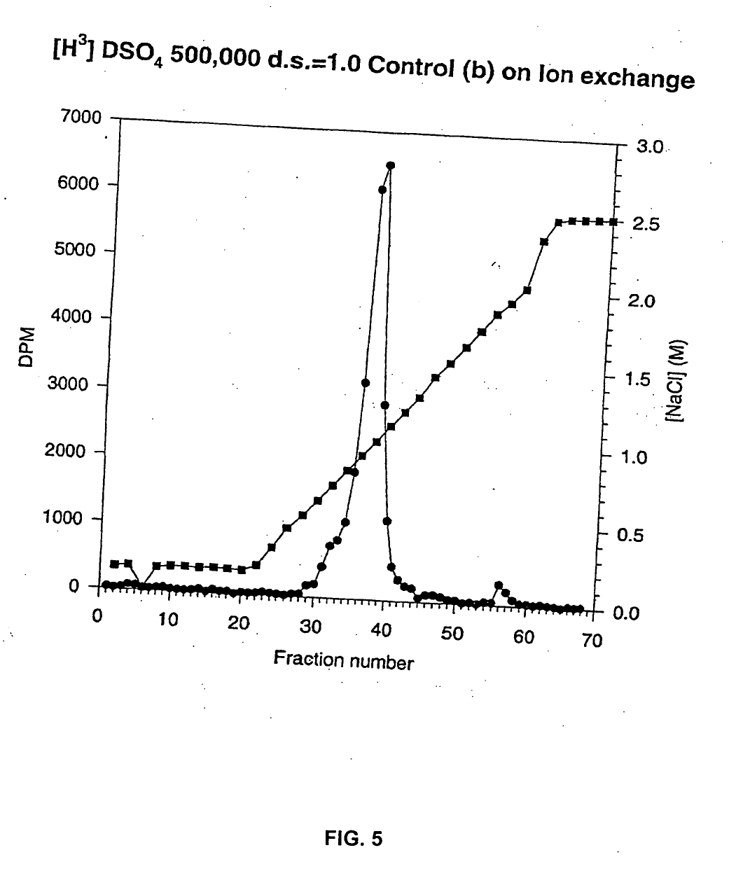

### 5. DETAILED DESCRIPTION OF THE INVENTION

In one embodiment of the invention, the inventor has discovered how to significantly increase the in vivo efficacy of certain sulfated polysaccharides against microbial infection, particularly viral infection, while reducing or avoiding adverse, unwanted or toxic effects of conventional sulfated polysaccharides. This is accomplished, in part, by controlling the percent of sulfation of the polysaccharide such that it is in the greater than 6% but below 13% range. Further, the invention also encompasses in an alternative embodiment controlling the molecular weight and/or percent of sulfation in order to obtain a sulfated polysaccharide with significant in vivo efficacy and without significant toxicity. The most preferred compositions or methods of the invention utilize sulfated α-1,6-linked polysaccharides or sulfated dextrans having the desired percent of sulfation and/or molecular weight which are flexible and thus useful against a wide variety of viruses. In a most preferred embodiment, the range of percent sulfation is effective to enable maximal interaction of constituent sulfate groups with the microbe which causes the infection, and wherein the sulfated polysaccharide is not substantially endocytosed or degraded by cell receptor binding in the mammal, and thereby retains antimicrobial activity in vivo.

The present inventor has also discovered that synthesizing lower sulfated polysaccharide or lowering the degree of charge density of sulfated polysaccharides, such as conventional dextran sulfate, eliminates or at least significantly reduces the binding and internalization of the sulfated polysaccharides by cell receptors for high charge density polyanions, for example in the kidney, and consequently eliminates or significantly reduces in vivo desulfation of these compounds. As a result, these sulfated polysaccharides having a low charge density retain their anti-microbial activity in vivo. This enables, for the first time, the systemic, topical, oral or rectal in vivo use in humans of stable sulfated polysaccharides, which have significant anti-microbial activity in vitro, to treat microbial disease or conditions.

Thus, the present invention encompasses methods for treating, preventing or managing microbial infections in vivo, particularly viral infections, bacterial infections, parasitic infections, or fungal infections with a sulfated polysaccharide or a pharmaceutically acceptable salt, hydrate, or stereoisomer thereof, having flexibility in its structure, a controlled degree of sulfation, and optionally homogeneity as to its molecular weight, and low degree of sulfation as compared to conventional dextran sulfate.

The present invention also provides methods for the treatment, prevention, or management of microbial infection comprising administering to a patient in need there of a therapeutically or prophylactically effective amount of a sulfated polysaccharide or pharmaceutically acceptable salts, hydrates, or stereoisomers thereof having from greater than 6% to below 13% sulfation. As mentioned above, such sulfated polysaccharides are particularly effective in the treatment of infectious diseases or conditions, including, but not limited to, viral infections, bacterial infection, parasitic infections, or fungal infections.

Without being limited by any theory, the sulfated polysaccharides and pharmaceutically acceptable salts, hydrates or stereoisomers thereof used in the methods or compositions of the invention have a percent sulfation sufficient for in vivo anti-microbial activity of the compound in a human, but which is controlled to enable the compound to escape binding by cell receptors for high charge density polyanions and desulfation after passage through the kidney. This results in retention of anti-microbial activity in vivo without toxicity or adverse effects.

Without being limited by any particular theory, the inventor believes that there is a range of charge density for sulfated polysaccharides within which they exhibit anti-microbial activity in vitro and retain their anti-microbial activity in vivo. In a preferred embodiment of the invention, the sulfated polysaccharides of the invention have a percent of sulfation of greater than 6% and below 13%, preferably greater than about 7% and below 13%, more preferably greater than about 8% and 12.5%, most preferably 9%, 9.5%, 10%, 10.5%, 11%, 11.5%, 12%, 12.2%, 12.5% or 12.8%, within ±1%.

A preferred sulfated polysaccharide used in the methods of the invention is sulfated dextran, or an α-1,6-linked polysaccharide, which has been modified to have the appropriate percent of sulfation. The sulfated dextran of the invention contain less than 13%, and may contain less than 12%, less than 11%, less than about 10%, less than 9%, less than 8%, and less than 7% sulfur, but more than 6% sulfur. In a preferred embodiment, the sulfated dextran variant has a sulfation of less than 13% and greater than 6%, more preferably, from about 7.0% to about 12.8%, even more preferably from about 8.5% to about 12.8%, and most preferably, from about 9.5% to less than 13%. Sulfated dextran having sulfation of about 12.2% and about 12.5% are particularly effective against retroviral infections.

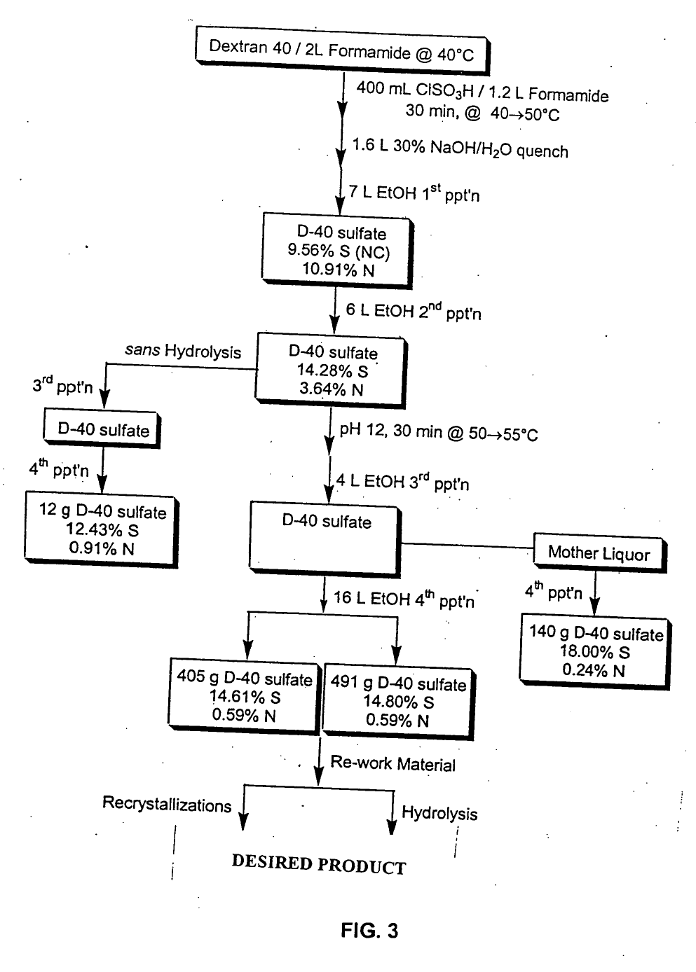

The sulfated polysaccharides of the invention, particularly the sulfated dextrans, can be prepared using known synthetic techniques and reagents. Several methods which are known in the art may be modified so that the proper degree of sulfation is achieved. These methods include those described in FIG. 3. However, as mentioned above, one may control the molecular weight as well as the degree of sulfation. Applicant has synthesized sulfated dextran with controlled sulphur contents and controlled degrees of sulfate substitution so that they are not taken up by cell receptors for highly charged polysaccharides. These polysaccharides exhibit essentially the same high antiviral activity in vivo as they do in vitro and have enhanced stability and longevity in vivo, as they are not readily taken up by cells they are also less toxic. Sulfated dextran, with controlled sulphur content is particularly suited as a viral cell attachment inhibitor because of its unique structure of essentially linear chain composed of an α-1,6-glycosidic linkage makes the most flexible of all polysaccharide chains that then enables maximal interaction of its constituent sulfate groups with positive charges on proteins of the virus and that it does not bind significantly to plasma proteins including albumin.

In another alternative embodiment, the invention encompasses the use of homogeneous sulfated polysaccharides. That is to say the sulfated polysaccharides administered in accordance with the methods described herein or utilized in the pharmaceutical compositions and dosage forms exhibit substantially the same percent of sulfation or molecular weight or both.

In a separate embodiment, the invention encompasses a method of treating or preventing a microbial infection in a mammal comprising administering to a mammal in need thereof a therapeutically effective amount of a composition comprising a sulfated polysaccharide having a percent of sulfate substitution per glucose residue in the polysaccharide ranging from greater than 6% to less than 13%, wherein the range of percent sulfation is effective to enable maximal interaction of constituent sulfate groups with the microbe which causes the infection, and wherein the sulfated polysaccharide is not substantially endocytosed or degraded by cell receptor binding in the mammal, and thereby retains antimicrobial activity in vivo. Preferably, the sulfated polysaccharide is sulfated dextran.

The invention also encompasses the treatment, prevention or management of anti-inflammatory diseases or disorders, interstitial cystisis and anti-arthritic diseases. The invention also encompasses the use of the sulfated polysaccharides of the invention as anti-albuminuric agents (albuminuria that occurs in kidney disease).

The invention further encompasses a method of treating or preventing a microbial infection in a mammal which comprises administering to a mammal in need thereof an effective amount of a levorotatory sulfated polysaccharide having a percent of sulfation from about 6% to about 20%; preferably from about 6% to about 13%; more preferably from about 9% to about 13%.

In a further embodiment, the invention encompasses a method of treating or preventing a microbial infection in a mammal which comprises administering to a mammal in need thereof an effective amount of a periodate-treated anionic polysaccharide. Preferably, the periodate treated anionic polysaccharide is a periodate treated sulfated dextran.

In another embodiment of the invention, the invention encompasses a method of treating or preventing a microbial infection in a mammal which comprises administering to a mammal in need of such treatment or prevention an effective amount of a co-charged anionic polysaccharide which has a percent of sulfation which enables maximal interaction with the microbe and which is not substantially endocytosed or degraded by cell receptor binding in the mammal thereby retaining antimicrobial in vivo. In a preferred embodiment, the co-charged anionic polysaccharide is co-charged with carboxymethyl groups, sulfonate groups, sulfate groups or mixtures thereof.

### 5.1. METHODS OF TREATMENT, PREVENTION AND MANAGEMENT OF MICROBIAL INFECTIONS

Viral infections which can be treated, prevented or managed by the methods of the present invention include, but are not limited to DNA and RNA viruses. The DNA and RNA viruses of the invention include, but are not limited to double-stranded DNA viruses, DNA reverse transcripting viruses, RNA reverse transcripting viruses, double-stranded RNA viruses, negative-sense single stranded RNA viruses, and positive-sense single-stranded RNA viruses. In particular, the methods and compositions are well suited for use against enveloped viruses. These include, for example, arenaviruses, enteroviruses, herpesviruses, myxoviruses, picornaviruses, poxviruses, retroviruses, rhabdoviruses, togaviruses. Specific double-stranded DNA viruses which can be treated, prevented or managed by the methods of the present invention include, but are not limited to, African swine fever virus (ASFV); BK virus (BKV); Bovine papillomavirus type 1 (BPV-1); Epstein-Barr virus (EBV); Human papillomavirus type 11 (HPV-11); Human papillomavirus type 40 (HPV-40); Pseudorabies virus (PrV) (Suid herpesvirus 1); Vaccinia virus (VV) (smallpox); and Varicella-zoster virus (VZV). Specific RNA reverse transcripting viruses which can be treated, prevented or managed by the methods of the present invention include, but are not limited to, Bovine immunodeficiency virus (BIV); Feline immunodeficiency virus (FIV); Feline leukemia virus (FeL V); HIV including Human immunodeficiency virus type 1 (HIV-1) and Human immunodeficiency virus type 2 (HIV-2); Human T-cell leukemia virus (HTLV-1); Murine leukemia virus (MLV); Rauscher murine leukemia virus; Simian immunodeficiency virus; and Simian type D retrovirus. Specific negative-sense single stranded RNA viruses which can be treated, prevented or managed by the methods of the present invention include, but are not limited to, Haemorrhagic septicaemia virus (VHSV); Influenza A virus; Influenza B virus; Junin virus; Lymphocytic choriomeningitis virus (LCM); Rabies; Respiratory syncytial virus (RSV); Sendai virus; Simian virus 40 (SV40); Tacaribe virus; and Vesicular stomatitis virus (VSV). Specific positive-sense single-stranded RNA viruses which can be treated, prevented or managed by the methods of the present invention include, but are not limited to, Classical swine fever virus (CSFV); Coxsackie virus B3; Cytomegalovirus (CMV); Echovirus 6; Foot-and-mouth disease virus (FMDV); Hepatitis A virus; Hepatitis C virus (HCV); Japanese encephalitis virus (JEV); Rubella virus (RV); Semliki forest virus; Sindbis virus;Transmissible gastroenteritis virus (TGEV) and Yellowfever virus (YFV).

Other viruses to be treated or prevented by the methods or compositions described herein include but are not limited to viruses that cause or are involved in cancer, hepatitis B, HSV-1, HSV-2, HCMV, MICMV, VZV, EBV, Measles Virus, Punto Toro a, VEE, West Nile virus, Vaccinia, Cow Pox, Adenovirus Type 1, Para Influenza Type 3, Pichinde and Rhinovirus Type 2. In another embodiment, the sulfated polysaccharides of the invention are used against drug resistant or multi-drug resistant strains of the above-mentioned viruses.

In a specific embodiment of the invention, the viruses to be treated are not HSV-1 or HSV-2.

Specific bacterium and parasites that may be treated, prevented or managed by the methods as described herein include, but are not limited to, Chlamydia trachomatis; Helicobacter pylori; Lactobacilli; Plasmodium sp.; Escherichia coli; Staphylococcus aureus; Staphylococcus epidermis; Staphylococcus hemolyticus; Saccharomyces cerevisiae; Pseudomonas aeruginosa; Legionella pneumophila; Neisseria gonorrhea; Neisseria meningitidis; Plasmodium knowlesi; and Plasmodium falciparum.

The present invention provides methods for introducing a therapeutically effective amount of a sulfated polysaccharide or combination of such sulfated polysaccharides into the blood stream, lymphatic system, and/or extracellular spaces of the tissue of a patient in the treatment and/or prevention of microbial infections, such as viral infection, bacterial infection or parasitic infection. The method comprises administering to a mammal at least sulfated polysaccharide that exhibits anti-microbial activity in vitro, the sulfated polysaccharide having a sulfation which results in retention of anti-microbial activity of the charged polysaccharide in vivo, e.g., sulfation that minimizes uptake by cells that have high charge-density cell receptors.

Without being limited by theory, the present inventor believes that the sulfated polysaccharides of the invention have a high affinity for the lymph nodes thus have and increased activity against viruses which populate or gestate in the lymphatic system. Thus, the present invention encompasses a method of administering a sulfated polysaccharide of the invention directly to or targeted for the lymphatic system of a patient.

The magnitude of a prophylactic or therapeutic dose of a sulfated polysaccharide of the invention or a pharmaceutically acceptable salt, solvate, hydrate, or stereoisomer thereof in the acute or chronic management of a disease, infection or condition will vary, however, with the nature and severity of the disease or infection, and the route by which the active ingredient is administered. The dose, and perhaps the dose frequency, will also vary according to the disease or infection to be treated, the age, body weight, and response of the individual patient. Suitable dosing regimens can be readily selected by those skilled in the art with due consideration of such factors. In one embodiment, the dose administered depends upon the specific compound to be used and the weight of the patient. In general, the dose per day is in the range of from about 0.001 to 500 mg/kg, preferably about 0.01 to 200 mg/kg, more preferably about 0.005 to 100 mg/kg. For treatment of human infections, about 0.1 mg to about 15 g per day is administered in about one to four divisions a day. Additionally, the recommended daily dose ran can be administered in cycles as single agents or in combination with other therapeutic agents. In one embodiment, the daily dose is administered in a single dose or in equally divided doses.

Different therapeutically effective amounts may be applicable for different diseases and infections, as will be readily known by those of ordinary skill in the art. Similarly, amounts sufficient to treat or prevent such diseases, but insufficient to cause, or sufficient to reduce, adverse effects associated with conventional therapies are also encompassed by the above described dosage amounts and dose frequency schedules.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of microbial infection. These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. Finally, the methods and compositions include the treatment of refractory patients or patients resistant to treatment such as resistance to reverse transcriptase inhibitors, protease inhibitors, etc.

5.1.1 Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, β-interferons, alkylating agents, hormones or cytokines.

The sulfated polysaccharides of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin,, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The sulfated polysaccharides of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The sulfated polysaccharides of the invention can be administered or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The sulfated polysaccharides of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

The sulfated polysaccharides of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The sulfated polysaccharides of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, pleconaril.

The sulfated polysaccharides of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114 (IDEC)) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The sulfated polysaccharides of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-alpha, and IFN-gamma).

The sulfated polysaccharides of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The sulfated polysaccharides of the invention can be administered or formulated in combination with β-interferons which include, but are not limited to, interferon beta-1a and interferon beta-1b.

The sulfated polysaccharides of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The compounds of the invention and the other therapeutics agent can act additively or, more preferably, synergistically. In a preferred embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent.

### 5.2. PERIODATE TREATED AND CO-CHARGED ANIONIC POLYSACCHARIDES

The invention encompasses sulfated polysaccharides that have been manipulated to reduce endocytosis by cell receptors and to increase the flexibility of the polysaccharide backbone to enable the efficient presentation of anionic charged groups to interact with regions on the targeted microbes.

One manipulation encompassed by the present invention is the treatment of sulfated polysaccharides with periodate. Periodate-treated anionic polysaccharides have increased flexibility due to periodate oxidation of some or all sugar residues. This treatment allows increased freedom of rotation and conformational flexibility of the polymers and provide flexible joints to facilitate biological interactions. Periodate-treated sulfated polysaccharides of the invention can have any counterion to ensure solubility including, but not limited to sodium, calcium, quaternary ammonium, and potassium.

Materials which may be periodate treated and used within the methods and compositions described herein also include the polysaccharides of Table I below.

Other variations include the incorporation of non-sulfate groups, such as carboxymethyl groups and sulfonate groups. By lowering the degree of substitution of charge on the polysaccharide with either sulfonate or carboxymethyl groups, the ability of the polysaccharide to be endocyctosed by high charge receptors is greatly reduced, therefore increasing its plasma stability. Carboxymethyl dextran sulfate can be prepared using a modification of methods of preparation employed by others (McLaughlin and Hirbst, 1950; Brown et al. 1964). Approximately 20 g of dextran is slurried in a mixture of isopropanol (350 ml) and 3.85M NaOH (40 ml) and is stirred for five minutes at 5° C. in a blender. Sodium chloroacetate (18 g) is added, and the whole mixture is stirred for 60 minutes at 5° C. under a nitrogen atmosphere, the mixture is removed from the blender and stored at 25° C. for three days. The degree of carboxymethyl substitution can be adjusted by varying the time at 25° C. from 1 day to 3 days as well as varying the mole ratio of CICH₂COONa to anhydroglucose from 1 to 4 and keeping the mola ratio of CICH₂COONa to NaOH to 1 to 1.4. After neutralisation the sample is washed with 80% ethanol and dried.

### 5.3. METHODS OF ACTIVATING SULFATED POLYSACCHARIDES FOR IN VIVO USE

In a separate embodiment, the invention encompasses a method of increasing or decreasing sulfation of naturally occurring sulfated polysaccharides for administration in vivo comprising providing the sulfated polysaccharide with a sulfation sufficient to eliminate or reduce binding of the sulfated polysaccharide by high charge density polyanion cell receptors and to provide anti-microbial activity to the sulfated polysaccharide. The sulfation range can be reached by preparation of compositions with the desired percent of sulfation. Alternatively, naturally occurring material can be controlled chemically or enzymatically to the degree of sulfation range wherein the sulfation is effective to enable maximal interaction of constituent sulfate groups with the microbe which causes the infection, and wherein the sulfated polysaccharide is not substantially endocytosed or degraded by cell receptor binding in the mammal, and thereby retains antimicrobial activity in vivo.

Listed in Table 1 below are examples of sulfated polysaccharides (not including dextran sulfate) whose anti-microbial activity has been demonstrated in vitro, but which previously have not been shown to have anti-microbial activity in vivo at a dosage below the cytotoxicity level of these compounds.

TABLE 1
Sulfated Polysaccharides having anti-viral or anti-bacterial activity in vitro

| Sulfated polysaccharides | In vitro activity |
|---|---|
| (14)-2-deoxy-2-sulfamido-3-O-sulfo-(14)-beta-D- | HIV |
| glycopyranan (derivative of chitosan) | |
| 2-acetamido-2-deoxy-3-O-sulfo(14)-beta-D- | HIV |
| glycopyranan (derivative of chitosan) | |
| Achranthese bidentata polysaccharide sulfate | HSV-1 |
| Aurintricarboxylic acid | HIV |
| Calcium spirulan | HIV, CMV, HSV-1, |
| | measles, mumps, |
| | influenza type A |
| Carboxymethylchitin | Friend murine |
| | leukemia virus, |
| | HSV |
| Chemically degraded heparin (Org 31733) | HIV, HHV-7 |
| Chondroitin polysulfate | HIV |
| Copolymer of sulphonic acid and biphenyl | HIV |
| disulphonic acid urea (MDL 10128) | |
| Curdlan sulfate | HIV, CMV |
| Cyanovirin-N (from cyanobacterium) | HIV |
| Fucoidin | HIV, Chlamydia, |
| | ASFV |
| Galactan sulfate | HIV, HSV-1 |
| Glucosamine-6-sulfate (monosaccharide) | HIV |
| Glycyrrhizin sulfate | HIV |
| Heparin | HIV, HHV-7, |
| | ASFV, Denge |
| | virus, MLV |
| Inositol hexasulfate | HIV |
| Lentinan sulfate | HIV |
| Mannan sulfate | HIV |
| N-acylated heparin conjugates | HIV |
| N-carboxymethylchitosan-N,O-sulfate | HIV, RLV |
| Oligonucleotide-poly(L-lysine)-heparin complexes | HIV |
| Pentosan polysulfate (xylanopolyhydrogen sulfate) | HIV, Chlamydia, |
| | ASFV |
| Peptidoglycan DS-4152 | HIV |
| Periodate degraded heparin | HIV |
| Phosphorothioate oligodeoxynucleotides | HIV |
| Polyacetal polysulfate | HIV |
| Polyinosinic-polycytidylic acid | HIV |
| Polysaccharides from Indocalamus tesselatus | HIV |
| (bamboo leaves) | |
| Prunellin | HIV |
| Rhamnan sulfate | HIV, HSV-1, CMV |
| Ribofuranan sulfate | HIV |
| Sodium lauryl sulfate | HIV, HSV |
| Sulfate dodecyl laminarapentaoside (alkyl | HIV |
| oligosaccharide) | |
| Sulfated bacterial glycosaminooglycan | HIV |
| Sulfated dodecyl laminari-oligomer (alkyl | HIV |
| oligosaccharide) | |
| Sulfated gangliosides | HIV |
| Sulfated laminara-oligosaccharide glycosides | HIV |
| synthesized from laminara-tetraose, laminara- | |
| pentaose, laminara-hexaose | |
| Sulfated N-deacetylatedchitin | Friend murine |
| | leukemia virus, |
| | HSV |
| Sulfated octadecyl maltohexaoside (alkyl | HIV |
| oligosaccharide) | |
| Sulfated octadecyl ribofurnans | HIV |
| Sulfated oligoxylan (heparin mimetic) | HIV |
| Sulfated xylogalactans | HIV-1 |
| Sulfatide (3′ sulfogalactosylceramide) | HIV |
| Sulfoevernan | HIV |
| Xylomannan sulfate | HIV, HSV-1, |
| | HSV-2 |

ASFV: African Swine Fever Virus;
HHV-7: Human Herpes Virus;
HSV: herpes simplex virus;
CMV: cytomegalovirus

Each of sulfated polysaccharides listed above, as well as any other sulfated polysaccharide that has anti-microbial activity in vitro, may be modified to bring their degree of sulfation or ionic charge to a level suitable for their use in the methods or compositions of the invention.

The invention further encompasses a method of treating or preventing a microbial infection in a mammal which comprises administering a compound chosen from the group consisting of cellulose sulfate; (14)-2-deoxy-2-sulfamido-3-O-sulfo-(14)-beta-D-glycopyranan (derivative of chitosan); 2-acetamido-2-deoxy-3-O-sulfo(14)-beta-D-glycopyranan (derivative of chitosan); Achranthese bidentata polysaccharide sulfate; Aurintricarboxylic acid; Calcium spirulan; Carboxymethylchitin; Chemically degraded heparin (Org 31733); Chondroitin polysulfate; Copolymer of sulphonic acid and biphenyl disulphonic acid urea (MDL 10128); Curdlan sulfate; Cyanovirin-N (from cyanobacterium); Fucoidin; Galactan sulfate; Glucosamine-6-sulfate (monosaccharide); Glycyrrhizin sulfate; Heparin; Inositol hexasulfate; Lentinan sulfate; Mannan sulfate; N-acylated heparin conjugates; N-carboxymethylchitosan-N,O-sulfate; Oligonucleotide-poly(L-lysine)-heparin complexes; Pentosan polysulfate (xylanopolyhydrogen sulfate); Peptidoglycan DS-4152; Periodate degraded heparin; Phosphorothioate oligodeoxynucleotides; Polyacetal polysulfate; Polyinosinic-polycytidylic acid; Polysaccharides from Indocalamus tesselatus (bamboo leaves); Prunellin; Rhamnan sulfate; Ribofuranan sulfate; Sodium lauryl sulfate; Sulfate dodecyl laminarapentaoside (alkyl oligosaccharide); Sulfated bacterial glycosaminooglycan; Sulfated dodecyl laminari-oligomer (alkyl oligosaccharide); Sulfated gangliosides; Sulfated laminara-oligosaccharide glycosides synthesized from laminara-tetraose, laminara-pentaose, laminara-hexaose; Sulfated N-deacetylatedchitin; Sulfated octadecyl maltohexaoside (alkyl oligosaccharide); Sulfated octadecyl ribofurrnans; Sulfated oligoxylan (heparin mimetic); Sulfated xylogalactans; Sulfatide (3′ sulfogalactosylceramide); Sulfoeveman; and Xylomannan sulfate, wherein the percent of sulfation of said compound has been controlled to enable maximal interaction of constituent sulfate groups with the microbe causing the infection, and wherein the compound is not substantially endocytosed or degraded by cell receptor binding in the mammal, thereby retaining antimicrobial activity in vivo.

### 5.4. PHARMACEUTICAL COMPOSITIONS AND DOSAGE FORMS

Pharmaceutical compositions and single unit dosage forms comprising a sulfated polysaccharide of the invention, or a pharmaceutically acceptable salt, hydrate or stereoisomer thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms of the invention typically also comprise one or more pharmaceutically acceptable excipients.

In an alternative embodiment, pharmaceutical composition encompassed by this embodiment include a sulfated polysaccharide of the invention, or a pharmaceutically acceptable salt, hydrate or stereoisomer thereof, and at least one additional therapeutic agent. Examples of additional therapeutic agents include, but are not limited to, those listed above in section 5.1.1.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise sulfated polysaccharides of the invention, or a pharmaceutically acceptable salt, hydrate, or stereoisomers thereof comprise 0.1 mg to 1500 mg per unit to provide doses of about 0.01 to 200 mg/kg per day.

5.4.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.4.2. Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

5.4.4. Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.5. Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). In a preferred embodiment of the invention, the sulfated polysaccharides of the invention have a molecular weight greater than about 500,000 when administered topically.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

5.4.6. Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1 990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free. The sulfated polysaccharides of the invention may also be administered directly to the lung by inhalation. For administration by inhalation, a sulfated polysaccharide can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a sulfated polysaccharide directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a sulfated polysaccharide to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a sulfated polysaccharide to the lung is a liquid spray device supplied, for example, by Aradigm Corporation. Liquid spray systems use extremely small nozzle holes to aerosolize liquid drug formulations that can then be directly inhaled into the lung.

In a preferred embodiment, a nebulizer device is used to deliver sulfated polysaccharides to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In a particularly preferred embodiment, an electrohydrodynamic (“EHD”) aerosol device is used to deliver sulfated polysaccharides to the lung. EHD aerosol devices use electrical energy to aerosolize liquid drug solutions or suspensions (see, e.g., Noakes et al., U.S. Pat. No. 4,765,539; Coffee, U.S. Pat. No., 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the sulfated polysaccharides formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of sulfated polysaccharides will be known to the skilled artisan and are within the scope of the invention.

Liquid drug formulations suitable for use with nebulizers and liquid spray devices and EHD aerosol devices will typically include a sulfated pulysaccharide with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of sulfated polysaccharide. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611, which are herein incorporated by reference) A sulfated polysaccharides can also be formulated in rectal or vaginal compositions such as suppositories or retention enemas, e.g., containing conventional suppository bases such as cocoa butter or other glycerides.

In addition to the formulations described previously, a sulfated polysaccharide can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver sulfated polysaccharides. Certain organic solvents such as dimethylsulfoxide can also be employed, although usually at the cost of greater toxicity. A sulfated polysaccharide can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem., 1983, 23, 61; see also Levy et al., Science, 1985, 228, 190; During et al., Ann. Neurol., 1989,25,351; Howard et al., 1989, J. Neurosurg. 71, 105). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g. Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4.7. Condoms and Prophylactic Devices

In a preferred embodiment of the invention, the sulfated polysaccharide can be used as a coating for a condom or other prophylactic device. Similarly, the sulfated polysaccharide can be used as a coating for surgical instruments and protective devices such as rubber gloves. When a sulfated polysaccharide of the invention is used as a coating as described herein, it is preferred to have a molecular weight higher than 500,000. The methods of using the sulfated polysaccharides of the invention as a coating will be well known by the skilled artisan. Similar methods can be found in U.S. Pat. No. 4,869,270 which is incorporated herein by reference.

5.4.8. Nutritional Products and Dietary Supplements

The sulfated polysaccharides may be incorporated into nutritional products including, but not limited to food compositions, over the counter, and dietary supplements. The sulfated polysaccharides may be added to various foods so as to be consumed simultaneously. As a food additive, the sulfated polysaccharides of the invention may be used in the same manner as conventional food additives, and thus, only needs to be mixed with other components to enhance the taste. Taste enhancement includes, but is not limited to, imparting to food a refreshingness, vitality, cleanness, fineness, or bracingness to the inherent taste of the food.

It will be recognized that dietary supplements may not use the same formulation ingredients or have the same sterile and other FDA requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements may take the form of tablets or capsules prepared by conventional means and optionally including other dietary supplements including vitamins, minerals, other herbal supplements, binding agents, fillers, lubricants, disintegrants, or wetting agents, as those discussed above. The tablets may be coated by methods well-known in the art. In a preferred embodiment, the dietary supplement may take the form of a capsule or powder to be dissolved in a liquid for oral consumption.

The amount of sulfated polysaccharides in a beverage or incorporated into a food product will depend on the kind of beverage, food and the desired effect. In general, a single serving comprises an amount of about 0.1% to about 50%, preferably of about 0.5% to about 20% of the food composition. More preferably a food product comprises sulfated polysaccharides in an amount of about 1% to about 10% by weight of the food composition.

Examples of food include, but are not limited to, confectionery such as sweets (candies, jellies, jams, etc.), gums, bean pastes, baked confectioneries or molded confectioneries (cookies, biscuits, etc.), steamed confectioneries, cacao or cacao products (chocolates and cocoa), frozen confectioneries (ice cream, ices, etc.), beverages (fruit juice, soft drinks, carbonated beverages), health drinks, health bars, and tea (green tea, black tea, etc.).

### 5.5. ASSAYS AND ANIMAL MODELS

The sulfated polysaccharides, compositions and dosage forms of the invention can be tested in vitro by a variety of methods known in the are to test antimicrobial activity. See, for example, the methods used throughout the examples

Generally, in vivo activity of a sulfated polysaccharide can be determined by directly administering the compound to a test animal, collecting blood samples and testing the blood for anti-microbial activity, for example. Standard models of in vivo antiviral activity include, but are not limited to, a primo-infection cynomolgus monkey model (Le Grand et al., Symp. Nonhum Primate Models AIDS. Sep. 19-22, 1993 11); and those described in The Handbook of Animal Models of Infection (Zak and Sande eds., Academic Press; 1st edition (1999), including but not limited to a Cytomegalovirus infections guinea pig model; a cytomegalovirus infection rat CMV model; a human cytomegalovirus infection of the SCID-hu (thy/liv) mouse model; an ocular cytomegalovirus infections in SCID-hu mice model; a simian varicella model; a varicella zoster infection of t-cells and skin in the SCID-hu mouse mode; a mouse model of influenza virus infection; a ferret model of influenza virus infection.; a cotton rat model of respiratory syncytial virus; a transgenic mouse models for HBV infections; a duck model for hepatitis B infection; a woodchuck model of hepatitis B virus infection; adult mouse models for rotavirus; a macaques model of SIV infection; a SCID-hu thy-liv mouse models for HIV infection; and a chimpanzee model of HIV-1 infection.

Standard models of in vivo antiparasitic activity include, but are not limited to, those described in The Handbook of Animal Models of Infection (Zak and Sande eds., Academic Press; 1st edition (1999), including but not limited to, an intravaginal mouse model of Trichomonas vaginalis infection.

Standard models of in vivo antifungal activity include, but are not limited to, those described in The Handbook of Animal Models of Infection (Zak and Sande eds., Academic Press; 1st edition (1999), including but not limited to, a Rodent model of candida sepsis; a generalized candida albicans infection model in the rat; a oropharyngeal and gastrointestinal candid a infection in mice model; a paw oedema model of localized candidiasis; a murine model of allergic bronchopulmonary aspergillosis; a pulmonary cryptococcus infection in mice model; a pulmonary cryptococcus neoformans infection in rats model; a rat model of invasive pulmonary aspergillosis; a rabbit model of candida keratomycosis; a rabbit model of cryptococcal meningitis; a rat models of ascending pyelonephritis due to candida albicans; a rat model of candida vaginal infection; and a murine model of candida vaginal infections.

Standard models of in vivo antibacterial activity include, but are not limited to, those described in The Handbook of Animal Models of Infection (Zak and Sande eds., Academic Press; 1st edition (1999)), including but not limited to, a mouse peritonitis/sepsis model; a murine thigh infection model; a mouse subcutaneous cotton thread model; a mouse peritonitis model; a murine models of peritonitis involving a foreign body; a rat polymicrobial peritonitis infection model; a mouse model of campylobacter jejuni infection; a suckling mouse model of enterotoxigenis escherichia coli infection; a rabbit model of shigellosis; the RITARD rabbit model of intestinal vibrio cholerne infections; a mouse model of helicobacter pylon infection; a ferret model of helicobacter; a hamster model of syphilis; a guinea pig model of acquired and congenital syphilis; a guinea pig model of legionnaires disease; a murine model of tuberculosis; a beige mouse model of disseminated mycobacterium avuim complex infection; an armadillo leprosy model; a mouse model of leprosy; a hamster model of lyme arthritis; a rabbit model of bacterial conjunctivitis; a murine model of bacterial keratitis; the rabbit intrastomal injection model of bacterial keratitis; a gerbil model of acute otitis media; a ginuea pig model of bacterial otitis extema; a chinchilla model of otitis media; a guinea pig model of acute otitis media; a rat model of bacterial epididymitis; a mouse model of mycoplasma genital infections; a mouse model of ascending urinary tract infection; a mouse model of ascending UTI involving short and long-term indwelling catheters; a rat model of subclinical pyelonephritis; a rat model of chronic cystitis; a mouse pneumococcal pneumonia model; a hamster model of mycoplasma pulmonary infections; a rat model of bacterial osteomyelitis of the tibia; a rat model of hematogenous osteomyelitis; a rabbit model of bacterial osteomyelitis of the tibia; a rat model of arthroplasty; a rabbit model of arthroplasty; a mouse model of streptococcal fasciitis; a rabbit model of bacterial endocarditis; an adult rat model of meningitis; and a rabbit model of bacterial meningitis.

In general, in vivo stability can be determined by a variety of models known to the skilled artisan. In particular, in vivo stability can be determined by a kidney perfusion assay. For either type of analysis, the test compound may be labeled, for example with tritium. A kidney perfusion technique is described in detail in Tay et al. (Am. J. Physiol., (1991), 260: F549-F554). Briefly, rat kidneys, e.g., from male Sprague-Dawley rats, are perfused with 5% bovine serum albumin (BSA) in modified Krebs Henseleit buffer containing amino acids and continually gassed with 95% O₂-5% CO₂. Samples that have been perfused may be subjected to ion-exchange chromatography using. for example, a 19×1/cm² column of sepharose Q. Samples are applied to the column in 6 M urea, 0.05 M Tris, 0.005% (w/v) Chaps, pH 7.0, and eluted with a linear gradient of 0.15-2.5 M NaCl in the same buffer at a flow rate of 0.5 ml/minute. Recoveries using this technique are very good.

The foregoing has demonstrated the pertinent and important features of the present invention. One of skill in the art will be appreciate that numerous modifications and embodiments may be devised. Therefore, it is intended that the appended claims cover all such modifications and embodiments.

### 6. WORKING EXAMPLES

The following examples are for the purpose of illustration only and are not intended as limiting the scope of the invention.

### 6.1 Example 1

### Synthesis of a Sulfated Dextran Having a Sulfation of 9.5%

Dextran T20(average molecular weight 20,000) was dried in vacuo at 60° C. overnight. The dried compound (100 g) was dissolved in 640 ml formnamide (FA). Chlorosulfonic acid (CSA) 80 ml was added to FA 200 ml at a maximum of 45° C. in a 3-necked flask, then cooled in ice-water. The amount of CSA determines the ultimate sulfation of the sulfated dextran (180 ml CSA to 200 ml FA yields approximately 17% sulfur). The CSA/FA mix was slowly added (over two hours) to the dextran at a temperature of 40° C. After all of the CSA/FA was added, the mixture was stirred for 15 minutes at a temperature of 45° C. The mixture was cooled to 25° C. and 28% NaOH was added slowly to give a pH 7.5-8.5 with a maximum temperature of 50° C. For the first precipitation, 3 L of ethanol were added with stirring. Stirring was stopped and the mixture was allowed to stand. The supernatant was decanted and the precipitate was redissolved in 1.5 L of water. For the second precipitation 1.5 L ethanol were added with stifling and then the mixture was allowed to stand for two hours. The supernatant was decanted and the precipitate was redissolved in 900 ml of water, to which 17 g NaCl was added. For the third precipitation 800 ml ethanol were added with stirring and the mixture was allowed to stand for two hours. The optical rotation-maximum was measured. The supernatant was decanted and the precipitate was redissolved in 500 ml water. 2.8 g Na₂HPO₄ and 2.6 g NaH₂PO₄ were added. For the final precipitation 5 L ethanol were added and the precipitate was filtered on a glass filter and dried in vacuo at 50° C.

### 6.2 Example 2

### Periodate Oxidation

Following the modified method of Smith degradation used by Sandy J D, Biochem J., 177: 569-574, 1979; chrondroitin sulfate (240 mg) was dissolved in 0.25M NaClO₄ (47 ml) at room temperature. 5 ml of 0.5 M NaIO₄ was added and KOH was used to adjust the mixture to pH 5. The reaction was allowed to proceed in the dark for 72 hours. The mixture was then dialysed in visking tubing to remove the periodate.

### 6.3 Example 3

### Introduction of Anionic Sulfur Groups to Carboxymethyl Dextran

Sulfated Form of Carboxymethyl Dextran (Average mw 20,000) with a Sulfur Content of 9.5%.

Carboxymethyl dextran (CMD) is dried in vacuo at 60° C. overnight. CMD (100 g) is dissolved in 640 ml formamide (FA). Chlorosulfonic acid (CSA) 80 ml is added to FA 200 ml at maximum of 45° C. in a 3-necked flask then cooled in ice-water. The amount of CSA will determine the ultimate sulfur content of CMD (180 ml OSA to 200 ml FA yields approx 17% sulfur). The CSA/FA mix is added slowly (over 2 hours) to CMD at a temperature of 40° C. After all is added the mixture is stirred for 15 minutes at a temperature of 45° C. The mixture is cooled to 25° C. and 28% NaOH is added slowly to give a pH 7.5-8.5 with a maximum temperature of 50° C. For the first precipitation, 3 L of ethanol is added with stirring. Supernatant is decanted and then residue is redissolved in 1.5 L of water. For the second precipitation 1.5 L ethanol is added with stirring and then allowed to stand for 2 hours. Supernatant is decanted and residue is redissolved in 900 ml of water and then added to 17 g NaCl. For the third precipitation 800 ml ethanol is add with stirring and allowed to stand for 2 hours. The optical rotation maximum should be 0.3. Supernatant is decanted and the residue is redissolved in 500 m water. Add 2.8 g Na₂HPO₄ and 2.6 g NaH₂PO₄. For the final precipitation 5 L ethanol is added and filtered on a glass filter and is dried in vacuo at 50° C.

Sulfonated Form of carboxymethyl dextran (average molecular weight 20,000).

Step 1. Dissolve 5 g dextran in water. Add 100 mg borohydride stir at room temp. for 30 min.
Step 2. Add sodium hydroxide pellets (10 g) and stir until dissolved and then sulfonate (12 g).
Step 3. Heat at 70° C. for 7 h. After 3 hours add a further 3 g of sulphonate. Continue heating for 4 hours.
Step 4. Neutralise with 5M HCl to pH 7.5 (Total volume(T)=75ml) and gradually add 200 ml ethanol with good stirring. Stop stirrer and stand 1 hour.
Step 5. Decant supernatant; redissolve in water (T=60 ml) and add 150 ml ethanol with good stirring. Stand 1 hour.
Step 6. Repeat as Step 5.
Step 7. Decant off the supernatant-redissolve the residue in 60 ml water and ppte in 600 ml ethanol. Some concentrated sodium chloride solution may be added to the mixture to aid precipitation.
Step 8. Filter and dry in vacuo. Yield approx. 6 g.

### 6.4 Example 4

### In Vivo Anti-Viral Activity

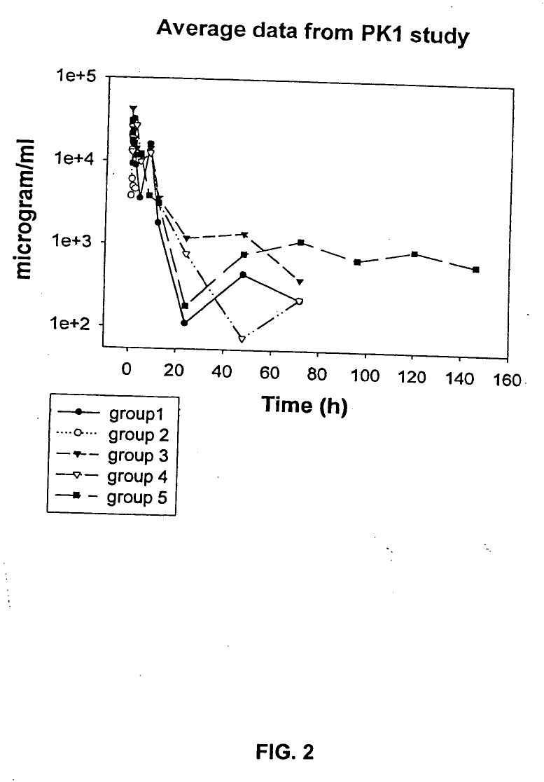

The in vivo anti-viral activity of dextran sulfate and variants of sulfated dextrans was assessed in a pharmacokinetic study involving single intravenous doses of 60 mg/kg commercially available (˜17% sulfur) dextran sulfate (DS) of 40,000 mw (group 1); DS 500,000 mw (group 2); dextran sulfate (12.2% sulfate)(DES6) 40,000 mw (group 3); DES6 500,000 mw (group 4) given to three male and three female rats and a multi-day injection of 60 mg/kg DES6, 500,000 mw given to an additional group of three rats (group 5). Rats were Sprague-Dawley, previously cannulated in the vena cava. Blood was drawn at various times after injection and assessed for anti-HIV activity in an acute infectivity cytoprotection assay system utilizing HIV-1 RF virus with CEN-SS cells using the MTS staining method for cell viability (based on Witvrouw et al., J. Acqur. Immun. Def. Syndr., 3:343-347, 1990). The results shown in FIG. 2 indicate that DS was, as expected, highly toxic at these doses with only one rat surviving beyond 24 hours. In contrast, good survival and circulating anti-HIV activity for as long as 120 hours after injection were observed in the DES6 treated rats.

FIG. 2 represents summary data from the five groups of animals. Each data point represents the concentration of circulating antiviral activity at times after injection. Concentration was calculated by determining the IC₅₀ of compound in the blood. As can be calculated from the raw data, DES6 of both molecular weights showed a prolonged half-life in the blood of between 12 and 18 hours, and an extended anti-viral activity (circulating concentration above the IC₅₀) beyond 72 hours. With three repeated injections of group 5 animals a steady state concentration was reached. Results are expressed in FIG. 2.

The data indicate that any mortality associated with DES6 was probably due to complications associated with the cannulation, since the MTD in non-cannulated animals is >850 mg/kg.

### 6.5 Example 5

### Effect on Pro-Thrombin/Thrombin and Activated Partial Thromboplastin Time

As noted above, inhibition of coagulation has been a repeatedly observed side effect of sulfated polysaccharide treatment, particularly with conventional dextran sulfate treatment. The purpose of this study was to evaluate the effects of DES6 compared to commercially available DS on prothrombin time (PT) and activated partial thromboplastin time (aPTT). All specimens were “spiked” with the test compound prior to submission to a Clinical Pathology Laboratory. The specimens were delivered along with reconstituted human plasma purchased from Sigma. Immediately prior to analysis 600 μl of the Sigma human plasma was added to each specimen.

A Bio-Merieux Coag-A-Mate MTX II Analyzer was used to measure Prothrombin Time (PT) and Activated Partial Thromboplastin Time (APTT). The PT reagent used was Simplastin L and the APTT reagent used was Platelin L; all reagents were obtained from Bio-Merieux. All specimens were run in duplicate. Coagulation control samples were analyzed immediately prior to testing.

| Parameter | Abbreviation | Units | Method |
|---|---|---|---|
| Prothrombin Time | PT | seconds | Photo-optical hemostasis |
| | | | analyzer |
| Activated partial | APTT | seconds | Photo-optical hemostasis |
| Thromboplastin | | | analyzer |
| Time | | | |

Specimen Disposition

No clotting times were obtained on several of the specimens. The PT measuring time started at five seconds and stopped at 60 seconds. The aPTT measuring time started at five seconds and stopped at 130 seconds. No clots were detected in these time frames. Results are presented in Table 2 below.

TABLE 2
Data Summary-Coagulation

| Sample No. | Clin Path ID | Sample Contents | | PT (sec) | APTT (sec) |
|---|---|---|---|---|---|
| PT Control 1 | Verify 1 | Range = 12.1-13.1 | | 12.5 | |
| PT Control 2 | Verify 2 | Range = 16.1-17.3 | | 16.7 | |
| APTT Control 1 | Verify 1 | Range = 25.8-29.6 | | | 27.5 |
| APTT Control | Verify 2 | Range = 47.3-54.1 | | | 51.5 |
| 1 | 0200059 | Plasma only | | 15.4 | 41.3 |
| 2 | 0200060 | 100 | μg/ml 8K DS | 15.3 | NC |
| 3 | 0200061 | 90 | μg/ml 8K DS | 15.1 | NC |
| 4 | 0200062 | 80 | μg/ml 8K DS | 14.9 | NC |
| 5 | 0200063 | 70 | μg/ml 8K DS | 15.3 | NC |
| 6 | 0200064 | 60 | μg/ml 8K DS | 15.4 | NC |
| 7 | 0200065 | 50 | μg/ml 8K DS | 15.1 | NC |
| 8 | 0200066 | 40 | μg/ml 8K DS | 15.6 | 60.2 |
| 9 | 0200067 | 30 | μg/ml 8K DS | 15.8 | 44.0 |
| 10 | 0200068 | 20 | μg/ml 8K DS | 15.8 | 45.3 |
| 11 | 0200069 | 10 | μg/ml 8K DS | 15.8 | 45.8 |
| 12 | 0200070 | 5 | μg/ml 8K DS | 15.6 | 55.2 |
| 13 | 0200071 | 1 | μg/ml 8K DS | 15.95 | 44.8 |
| 14 | 0200072 | 0.1 | μg/ml 8K DS | 16.0 | 43.8 |
| 15 | 0200073 | 100 | μg/ml 40K DS | 42.5 | NC |
| 16 | 0200074 | 90 | μg/ml 40K DS | 15.6 | NC |
| 17 | 0200075 | 80 | μg/ml 40K DS | 14.7 | NC |
| 18 | 0200076 | 70 | μg/ml 40K DS | 14.1 | 59.4 |
| 19 | 0200077 | 60 | μg/ml 40K DS | 13.9 | 86.3 |
| 20 | 0200078 | 50 | μg/ml 40K DS | 14.2 | 100.6 |
| 21 | 0200079 | 40 | μg/ml 40K DS | 14.5 | 59.0 |
| 22 | 0200080 | 30 | μg/ml 40K DS | 15.6 | 46.9 |
| 23 | 0200081 | 20 | μg/ml 40K DS | 15.9 | 45.4 |
| 24 | 0200082 | 10 | μg/ml 40K DS | 14.0 | 45.8 |
| 25 | 0200083 | 5 | μg/ml 40K DS | 13.8 | 106.6 |
| 26 | 0200084 | 1 | μg/ml 40K DS | 16.6 | 46.1 |
| 27 | 0200085 | 0.1 | μg/ml 40K DS | 16.7 | 46.2 |
| 28 | 0200086 | 100 | μg/ml 500K DS | 16.0 | 47.3 |
| 29 | 0200087 | 90 | μg/ml 500K DS | 15.7 | 47.95 |
| 30 | 0200088 | 80 | μg/ml 500K DS | 15.8 | 47.8 |
| 31 | 0200089 | 70 | μg/ml 500K DS | 16.1 | 47.5 |
| 32 | 0200090 | 60 | μg/ml 500K DS | 16.0 | 48.0 |
| 33 | 0200091 | 50 | μg/ml 500K DS | 16.8 | 46.9 |
| 34 | 0200092 | 40 | μg/ml 500K DS | 16.9 | 46.8 |
| 35 | 0200093 | 30 | μg/ml 500K DS | 16.8 | 46.7 |
| 36 | 0200094 | 20 | μg/ml 500K DS | 16.9 | 46.7 |
| 37 | 0200095 | 10 | μg/ml 500K DS | 16.5 | 47.0 |
| 38 | 0200096 | 5 | μg/ml 500K DS | 17.8 | NC |
| 39 | 0200097 | 1 | μg/ml 500K DS | 17.0 | 47.0 |
| 40 | 0200098 | 0.1 | μg/ml 500K DS | 16.9 | 47.2 |
| 41 | 0200099 | 100 | μg/ml 40K Des 6 | 15.6 | 51.5 |
| 42 | 0200100 | 90 | μg/ml 40K Des 6 | 16.2 | 51.9 |
| 43 | 0200101 | 80 | μg/ml 40K Des 6 | 15.0 | 62.4 |
| 44 | 0200102 | 70 | μg/ml 40K Des 6 | 15.0 | 63.8 |
| 45 | 0200103 | 60 | μg/ml 40K Des 6 | 15.3 | 60.9 |
| 46 | 0200104 | 50 | μg/ml 40K Des 6 | 14.7 | 87.5 |
| 47 | 0200105 | 40 | μg/ml 40K Des 6 | 14.7 | 98.9 |
| 48 | 0200106 | 30 | μg/ml 40K Des 6 | 14.7 | 85.5 |
| 49 | 0200107 | 20 | μg/ml 40K Des 6 | 14.2 | 75.5 |
| 50 | 0200108 | 10 | μg/ml 40K Des 6 | 16.9 | 49.4 |
| 51 | 0200109 | 5 | μg/ml 40K Des 6 | 21.4 | NC |
| 52 | 0200110 | 1 | μg/ml 40K Des 6 | 15.1 | 62.1 |
| 53 | 0200111 | 0.1 | μg/ml 40K Des 6 | 16.7 | 50.6 |
| 54 | 0200112 | 100 | μg/ml 500K Des 6 | 17.4 | 50.0 |
| 55 | 0200113 | 90 | μg/ml 500K Des 6 | 17.5 | 48.8 |
| 56 | 0200114 | 80 | μg/ml 500K Des 6 | 17.2 | 52.2 |
| 57 | 0200115 | 70 | μg/ml 500K Des 6 | 17.6 | 49.2 |
| 58 | 0200116 | 60 | μg/ml 500K Des 6 | 17.6 | 49.1 |
| 59 | 0200117 | 50 | μg/ml 500K Des 6 | 17.5 | 51.1 |
| 60 | 0200118 | 40 | μg/ml 500K Des 6 | 17.5 | 50.2 |
| 61 | 0200119 | 30 | μg/ml 500K Des 6 | 17.6 | 50.1 |
| 62 | 0200120 | 20 | μg/ml 500K Des 6 | 17.6 | 49.6 |
| 63 | 0200121 | 10 | μg/ml 500K Des 6 | 17.6 | 50.4 |
| 64 | 0200122 | 5 | μg/ml 500K Des 6 | 16.6 | 55.2 |
| 65 | 0200123 | 1 | μg/ml 500K Des 6 | 17.7 | 49.4 |
| 66 | 0200124 | 0.1 | μg/ml 500K Des 6 | 17.6 | 49.5 |
| 67 | 0200125 | 1.5 | μL K DMSO | 17.7 | 49.8 |
| 68 | 0200126 | 1.35 | μL K DMSO | 17.6 | 49.7 |
| 69 | 0200127 | 1.2 | μL K DMSO | 17.6 | 49.7 |
| 70 | 0200128 | 1.05 | μL K DMSO | 17.6 | 49.7 |
| 71 | 0200129 | 0.9 | μL K DMSO | 17.7 | 49.7 |
| 72 | 0200130 | 0.75 | μL K DMSO | 17.8 | 50.1 |
| 73 | 0200131 | 0.6 | μL K DMSO | 17.8 | 50.1 |
| 74 | 0200132 | 0.45 | μL K DMSO | 17.9 | 49.95 |
| 75 | 0200133 | 0.3 | μL K DMSO | 17.8 | 50.0 |
| 76 | 0200134 | 0.15 | μL K DMSO | 17.9 | 49.95 |
| 77 | 0200135 | 0.08 | μL K DMSO | 17.9 | 50.1 |
| 78 | 0200136 | 0.015 | μL K DMSO | 18.0 | 49.8 |
| 79 | 0200137 | 0.0015 | μL K DMSO | 17.9 | 49.8 |

Thrombocytopenia and Coagulation

Experiments to determine the effect of injected DS and DES6 of various molecular weights on coagulation parameters were undertaken. Rats were given either 5 or 50 mg/kg (i.v.) of each compound on consecutive days for ten days. On day 11, dosages were changed from 5 to 1 mg/kg and 50 to 100 mg/kg and daily consecutive intravenous injections were continued. At days 0, 5, 10 and 15 blood was drawn and assessed for aPTT and platelet counts. Results are provided in Table 3 below.

TABLE 3

| Parameter | APTT (seconds) | | | | PLATELET (K/μl) | | | |
|---|---|---|---|---|---|---|---|---|
| Animal | Day | | | | Day | | | |
| Number | 5 | 10 | 15 | 20 | 5 | 10 | 15 | 20 |

Group 1 - 8000K Dextran Sulfate - 5 mg/kg

| | | | | | | | | |
|---|---|---|---|---|---|---|---|---|
| 12030 | 60.5 | 50.0 | 21.6 | 12.77 | 1370 | 1813* | 1466 | Clotted |
| 12031 | 51.4 | 50.4 | 22.0 | >130 | 1118 | 1424 | 1277 | 1380 |
| 12032 | 50.8 | 50.7 | 21.6 | 12.91 | 1256 | 1483 | 1161 | 1185 |

Group 2 - 8000K Dextran Sulfate - 50 mg/kg

| | | | | | | | | |
|---|---|---|---|---|---|---|---|---|
| 12033 | >130 | >130 | >130 | >130 | 1181 | 965 | 684 | 1554 |
| 12034 | >130 | >130 | >130 | >130 | 1312 | 1182* | 1010 | 1350 |
| 12035 | >130 | >130 | >130 | >130 | 1328 | 1182 | 749 | 1834 |

Group 3 - 500K Des 6 - 5 mg/kg

| | | | | | | | | |
|---|---|---|---|---|---|---|---|---|
| 12036 | 57.8 | 60.0 | 16.0 | 14.16 | 1250 | 1206* | 1240 | 1156 |
| 12037 | 64.3 | 70.7 | 17.0 | 15.87 | 1155 | 1242 | 1143 | 1196 |
| 12038 | 60.1 | 72.4 | 16.7 | 17.22 | 1164 | 1283 | 1050 | 1094 |

Group 4 - 500K Des 6 - 50 mg/kg

| | | | | | | | | |
|---|---|---|---|---|---|---|---|---|
| 12039 | >130 | >130 | >130 | >130 | 1176 | 1167 | 1133 | 920 |
| 12040 | >130 | >130 | >130 | >130 | 1110 | 940 | 797 | 1126 |
| 12041 | >130 | >130 | >130 | 87.55 | 912 | 966 | 760 | 696 |

Group 5 - Baseline Blood Profile - Day 0

| | | |
|---|---|---|
| 12042 | 14.3 | 1104 |
| 12043 | 15.7 | 1221 |
| 12044 | 12.6 | 1291 |

Value flagged for platelet clumping; smears evaluated and no clumping seen.

Maximum Tolerated Dose

The multiple toxicity dose (MTD) of DES6 was assessed in a series of experiments where groups of five rats were given 100 or 200 mg/kg doses of DES6 mw=500,000. Body weights and overall behavioral assessments were determined for five days after injection. There were no overt signs of toxicity as determined by observation and body weight measurements. Subsequently rats were given a 500 mg/kg injection and observed for a further five days also without signs of toxicity. Finally animals were given a dose of 850 mg/kg. Results are provided below in Table 4.

TABLE 4
MAXIMUM TOLERATED DOSE (MTD)

| | Average body weight (n = 5) | S.D |
|---|---|---|
| 200 mg/kg | | |
| Day 1 | 277.4 | 15.9 |
| 2 | 277.9 | 13.9 |
| 3 | 288.9 | 14.9 |
| 4 | 294.4 | 15.2 |
| 5 | 296.0 | 22.3 |
| 6 | 300.1 | 25.4 |
| 500 mg/kg | | |
| Day 7 | 328.6 | 21.9 |

### 6.6 Example 6

### In vitro Anti-Viral Assessment of Sulfated Polysaccharides

The studies included assessment of five test compounds at a high test concentration of 500 μg/ml in human peripheral blood mononuclear cells (PBMCs).

Methods

All test compounds #3 (dextran sulfate 17-20%), #4 (sulfated dextran, 9.5% sulfur, molecular weight 30,000), and #6 (sulfated dextran, 12.2% sulfur, molecular weight 36,000) were solubilized in H₂O at 40 mg/ml. The compounds were visually completely soluble and colorless. Compounds were light protected and assays were performed in a manner which minimized incidental light. Compounds were stored at −20° C. following solvation.

Viruses

The low passage pediatric isolate RoJo was derived in the laboratories of Southern Research Institute. RoJo is a presumed subtype B virus.

PBMC Isolation and Blasting

Peripheral blood monocular cells (PBMCs) were obtained from normal hepatitis and HIV-1 negative donors by ficoll hypaque gradient separation. The mononuclear cells were washed to remove residual separation media, counted, viability determined and resuspended in RPMI 1640 medium supplemented with 15% FBS (heat inactivated), 2 mM L-glutamine, 100 U/mL penicillin, 100 μg/mL streptomycin, and 10 μg/mL gentamycin with 2 μg/mL phytohemagluttin (PHA) at 1×10⁶ cells/mL. The cells were cultured for 48 to 72 h at 37° C., 5% CO₂. Following incubation, cells were collected by centrifugation, washed and resuspended in RPMI 1640 supplemented with 15% FBS (heat inactivated), 2 mM L-glutamine, 100 U/mL penicillin, 100 μg/mL streptomycin, and 10 μg/mL gentamycin with 20 U/mL recombinant IL-2 R & D Systems, Minneapolis, Minn.). IL-2 was included in the culture medium to maintain the cell division initiated by the PHA mitogenic stimulation. The cultures were then maintained until use by ½ culture volume change with fresh IL-2 containing medium every three days.

PBMC Assay

Human peripheral blood mononuclear cells from a minimum of two donors, that have been blasted with PHA and IL-2, were counted, viability determined by Trypan Blue dye exclusion and mixed in equal ratios. Pooled donors were used to minimize the variability observed between individual donors which results from quantitative and qualitative differences in HIV infection and overall response to the PHA and IL-2 of primary lymphocyte populations. The cells were resuspended at 1×10⁶ cells /mL in RPMI 1640 without phenol red supplemented with 15% Fetal Bovine Serum (heat inactivated), 2 mM L-glutamine, 100 U/mL penicillin, 100 μg/mL streptomycin, 10 μg/mL gentamycin and IL-2 (20 U/mL, R&D Systems, Minneapolis, Minn.). Fifty microliters of cells were then distributed to the inner 60 wells of a 96 well round bottom microtiter culture plate in a standard format developed by the Infectious Disease Research department of Southern Research Institute. Each plate contains cell control wells (cells only), virus control wells (cells plus virus), and experimental wells (drug plus cells plus virus). Serially diluted compounds were added to the microtiter plate followed by the appropriate pretitered dilution of HIV-1 RoJo. All samples were assayed in triplicate with a replicate plate without virus for the determination of compound toxicity. The final volume per well was 200 μL. The assay was incubated for 6 days in a humidified atmosphere at 37° C., 5% CO₂, after which supernatants were collected, for analysis of RT activity and sister plates analyzed for cell viability by MTS dye reduction. Wells were also examined microscopically and any abnormalities noted.

MTS Staining for Cell Viability

At assay termination the assay plates were stained with the soluble tetrazolium-based dye MTS (CellTiter96® Reagent Promega) to determine cell viability and quantify compound toxicity. MTS is metabolized by the mitochondria enzymes of metabolically active cells to a soluble formazan product, allowing the rapid quantitative analysis cell viability and compound cytotoxicity. This reagent is a single stable solution that does not require preparation before use. At termination of the assay 20 μL of MTS reagent was added per well and incubated for 4 h at 37° C. Adhesive plate sealers were used in place of the lids, the sealed plate was inverted several times to mix the soluble formazan product and the plate was read spectrophotometrically at 490 nm with a Molecular Devices Vmax plate reader.

Reverse Transcriptase Assay for Culture Supernatants

Reverse transcriptase (RT) activity was measured in cell-free supernatants. Tritiated thymidine triphosphate (NEN) (TTP) was resuspended in distilled H₂O at 5 Ci/mL. Poly rA and oligo dT were prepared as a stock solution which was kept at −20° C. The RT reaction buffer was prepared fresh on a daily basis and consists of 125 μL 1.0 M EGTA, 125 μL dH₂O, 110 μL 10% SDS, 50 μL 1.0 M Tris (pH 7.4), 50 μL 1.0 M DTT, and 40 μL 1.0 M MgCl₂. These three solutions were mixed together in a ratio of two parts TTP, one part poly rA:oligo dT, and one part reaction buffer. Ten microliters of this reaction mixture were placed in a round bottom microtiter plate and 15 μL of virus containing supernatant was added and mixed. The plate was incubated at 37° C. in a water bath with a solid support to prevent submersion of the plate and incubated for 60 minutes. Following reaction, the reaction volume was spotted onto pieces of DE81 paper, washed 5 times for 5 minutes each in a 5% sodium phosphate buffer, two times for one minute each in distilled water, two times for one minute each in 70% ethanol, and then dried. Opti-Fluor O was added to each sample and incorporated radioactivity was quantitated utilizing a Wallac 1450 Microbetaplus liquid scintillation counter.

Data Analysis

IC₅₀ (50%, inhibition of virus replication), TC₅₀ (50% reduction in cell viability) and a therapeutic index (TI, TC₅₀/IC₅₀) are provided.

Results

The IC₅₀ and TC₅₀ values were calculated by linear regression. The TI represents the ratio of the TC₅₀/IC₅₀, and is used to determine relative potency between compounds. The graphical representation shows the relationship between antiviral efficacy (% VC) and compound toxicity (% CC) expressed as a percent of the control, virus no compound or cells no compound, respectively.

All PBMC assays used to evaluate the test compounds met the individual assay standards and internal assay validation criteria including intra-triplicate variation and total virus replication. The control compounds AZT (RT inhibitor) and conventional dextran sulfate (virus entry/attachment inhibitor) inhibited HIV replication with the expected efficacies (AZT: IC₅₀ 1 to 10 nM; dextran sulfate: IC₅₀ 0.1 to 2 μg/ml). Thus the presented evaluations are valid and representative of the antiviral activity of the tested compounds.

The data are summarized in Table 5.

TABLE 5
Antiviral Activity

| | | Antiviral Efficacy | | |
|---|---|---|---|---|
| Compound | Assay | IC₅₀ | TC₅₀ | TI |
| DES 17-20% Sulfation | 1 | 1.0 | >100 | >100 |
| (μg/ml) | 2 | 0.5 | >500 | >926 |
| DES 9.5% Sulfation | 1 | 19.4 | >500 | >26 |
| (μg/ml) | | | | |
| DES 12.5% Sulfation | 1 | 1.6 | >500 | >317 |
| (μg/ml) | | | | |
| AZT (μM) | 1 | 0.002 | >1 | >386 |
| | 2 | 0.005 | >1.0 | >185 |
| DextranSulfate 17-20% | 1 | 1.1 | >100 | >89 |
| (μg/ml) | 2 | 1.8 | >100 | >57 |

Table 5 compares the previous and current antiviral evaluations in PBMCs. The previously identified IC₅₀ and antiviral efficacy of DES 17-20% Sulfation was verified with an IC₅₀ of 0.5 μg/ml in these experiments. This is within the standard 3-fold error predicted for the PBMC assay. In addition, the second experiment demonstrated that compound #3 is non-cytotoxic to PBMCs at 500 μg/ml.

In this set of evaluations the initial antiviral assessments of DES 9.5% Sulfation and DES 12.5% Sulfation were performed. Both compounds were non-cytotoxic at 500 μg/ml and 50% inhibitory concentrations were derived. DES 12.5% Sulfation displayed antiviral activity equivalent to DES 17-20% Sulfation based upon the calculated IC₅₀, 1.6 vs. 0.5 μg/ml, respectively. Additionally, examination of the antiviral efficacy curves suggests that the 2 compounds are of equal potency. In contrast, DES 9.5% Sulfation was 39-fold less active than DES 17-20% Sulfation and 12-fold less active than DES 12.5% Sulfation.

### 6.7 Example 7

### In vitro Anti-Viral Assessment of Sulfated Polysaccharides

The following compounds have been tested for in vitro anti viral activity. Sample 3(dextran sulfate, 17-20% sulfur, molecular weight 39,700), sample 4 (dextran sulfate, 9.5% sulfur, molecular weight 30,000) and sample 6 (dextran sulfate, 12.2% sulfur, molecular weight 36,000). All three compounds exhibited significant anti-viral activity against HIV-1 RoJo virus.

DES 9.5% Sulfation and DES 12.5% Sulfation were also assessed against a range of HIV-1 clinical isolates, including subtype representative isolates, SIV and HIV-2. The inhibition of HIV-1 ADA and BaL replication in monocyte/macrophages was also assessed.

DES 9.5% Sulfation and DES 12.5% Sulfation were prepared as described above in Example 4.

Viruses

Human immunodeficiency virus type 1 (HIV-1) strains Ba-L, ADA, SIVmac251, HIV-2 (CDC3 10319) and the subtype representative strains (Table 6) were obtained from the NIAID AIDS Research and Reference Reagent Program. The low passage pediatric isolates SLKA, WeJo and TeKi were derived in the laboratories of Southern Research Institute. The multi-drug resistant virus MDR-769 was derived from a highly experienced antiretroviral patient and exhibits the resistance profile and genotype outlined in Table 7.

TABLE 6
Subtype Representative Viruses

| | Subtype |
|---|---|
| Virus | (env) |
| RW/92/016 | A |
| 302056 (91US056) | B |
| BR/92/025 | C |
| UG/92/046 | D |
| CMU02 | E |
| BR/93/020 | F |
| Jv1083 | G |
| BCOF-01 | O |

TABLE 7
Phenotype and Genotype of the MDR 769 Virus

| | | Other Changes from | Drug |
|---|---|---|---|
| Gene | Resistance Mutations | Consensus B | Resistance |
| RT | M41L, K65R, D67N | K20R, V21I, V35I, K43Q, | AZT, ddI, |
| | V751, F116Y, Q151M, | A62V, E79D, I167I/V, | 3TC, d4T, |
| | Y181I, L210W, T215Y | G196E, Q197K, E207Q, | PFA, NVP |
| | | D218E | |
| PR | L10I, M36M/V, M46I, | V13I, D60E, I62V, K223Q | IDV, |
| | I54V, L63P, A71V, | | SQV, |
| | V82A, I84V, L90M | | NFV |

Mutations in bold face type in Table 7 represent key resistance mutations in the indicated genes.
PBMC Isolation and Blasting

Peripheral blood monocular cells (PBMCs) were obtained as described in Example 6.

PBMC Assay

PBMC assays were carried out as described in Example 6.

Monocyte Isolation Culture and Infection

Peripheral blood monocytes were isolated from normal HIV-1 negative donors by plastic adherence following ficoll hypaque purification of the buffy coat, as described above for PBMCs. In many cases the same donor used to produce the PBMC populations was also used to produce monocyte/macrophages, however unlike PBMC population monocyte/macrophage, donors were never pooled. Following a two hour adherence in RPMI 1640 without phenol red supplemented with 10% human pooled AB serum (heat inactivated), 2 mM L-glutamine, 100 U/mL penicillin, 100 μg/mL streptomycin, 10 μg/mL gentamycin, cultures were washed to remove non-adherent cells. The monocytes were released from the plastic by vigorous pipetting with Ca²⁺ and Mg²⁺ free PBS. Adherent cells were assessed for purity by nonspecific esterase staining (a-napthyl butyrate specific esterase, Sigma Chemical Co.), and/or viability by Trypan Blue dye exclusion, counted and resuspended in RPMI 1640 supplemented with 10% Fetal Bovine Serum (heat inactivated), 2 mM L-glutamine, 100 U/mL penicillin, 100 μg/mL streptomycin, 10 μg/mL gentamycin at 1×10⁶ monocytes per ml. The monocytes (1×10⁵ per 0.2 cm well) were then cultured for six days, allowing maturation of the cells to a macrophagelike phenotype. At day six the cultures were washed three times to remove any non-adherent cells and serially diluted test compounds added followed by the addition of a pre-titered amount of HIV-1 virus, if microscopic observation of the wells demonstrated a 70% or greater confluency of the monocyte/macrophage monolayer. Cultures were washed a final time by media removal 24 hours post infection, fresh compound added and the cultures continued for an additional six days. The assays were preformed using a standardized microtiter plate format, which uses only the inner 60 wells of a 96 well plate for assay purposes. The outer rows contain media and acts an a evaporation barrier. Each plate contains cell control wells (cells only), virus control wells (cells plus virus), and experimental wells (drug plus cells plus virus). HIV p24 antigen content to assess virus replication was measured at assay termination by a commercially available p24 ELISA assay (Coulter) on cell-free supernatants, and compound cytotoxicity by MTS dye reduction. AZT, HIV-1 reverse nucleoside transcriptase inhibitor and dextransulfate, an attachment inhibitor, were used as positive control compounds and run in parallel with each determination. At termination of the assay culture plates were removed from the incubator and observed microscopically. Any unique findings were noted.

MTS Staining for Cell Viability

MTS staining was carried out as described in Example 6.

Reverse Transcriptase Assay fur Culture Supernatants

Reverse transcriptase (RT) activity was measured in cell-free supernatants as described in Example 6.

P24 Antigen ELISA

ELISA kits were purchased from Coulter Electronics. The assay was performed according to the manufacturer's instructions. Control curves were generated in each assay to accurately quantitate the amount of p24 antigen in each sample. Data were obtained by spectrophotometric analysis at 450 nm using a Molecular Devices Vmax plate reader. Final concentrations were calculated from the optical density values using the Molecular Devices Soft Max software package.

Results

IC₅₀ (50%, inhibition of virus replication), TC₅₀ (50% reduction in cell viability) and a therapeutic index (TI, TC₅₀/IC₅₀) were calculated. The results are summarized in Table 8.

The antiviral data for each test include the relevant raw data values from the triplicate tests for virus replication (RT (cpm) for PBMCs and p24 (pg/ml) for monocytes) and cell viability (OD 490) for MTS dye reduction. The IC₅₀ and TC₅₀ values were calculated by linear regression. The TI represents the ratio of the TC₅₀/IC₅₀, and is used to determine relative potency between compounds. The graphical representation shows the relationship between antiviral efficacy (% VC) and compound toxicity (% CC) expressed as a percent of the control, virus no compound or cells no compound, respectively.

TABLE 8
Summary of the Range of Action Testing

| | | DES 9.5% Sulfation | | | DES 12.5% | | | | | |
|---|---|---|---|---|---|---|---|---|---|---|
| | | (μg/ml) | | | Sulfation (μg/ml) | | | AZT (μM) | | |
| Virus | Cells | IC₅₀ | TC₅₀ | TI | IC₅₀ | TC₅₀ | TI | IC₅₀ | TC₅₀ | TI |
| Subtype A | BMC | 6.4 | 100 | 1 | .7 | 100 | 15 | .001 | 1 | 1000 |
| RW/92/016 | | | | | | | | | | |
| Subtype B | BMC | 9.7 | 100 | 2.5 | .8 | 100 | 120 | .01 | 1 | 100 |
| 302056 | | | | | | | | | | |
| (91US056) | | | | | | | | | | |
| Subtype C | BMC | 6.5 | 100 | 6.1 | 0.3 | 100 | 9.7 | .005 | 1 | 200 |
| BR/92/025 | | | | | | | | | | |
| Subtype D | BMC | 7.8 | 100 | 1.5 | .9 | 100 | 54 | .001 | 1 | 1000 |
| UG/92/046 | | | | | | | | | | |
| Subtype E | BMC | 0.5 | 100 | 9.5 | .7 | 100 | 149 | .003 | 1 | 333 |
| CMU02 | | | | | | | | | | |
| Subtype F | BMC | 0 | 100 | 5 | .3 | 100 | 323 | .003 | 1 | 333 |
| BR/93/020 | | | | | | | | | | |
| Subtype G | BMC | 100 | 100 | — | 1.7 | 100 | 9 | .006 | 1 | 167 |
| Jv1083 | | | | | | | | | | |
| Subtype O | BMC | 8.4 | 100 | 1 | .1 | 100 | 95 | .003 | 1 | 333 |
| BCOF-01 | | | | | | | | | | |
| RoJo | BMC | 6 | 100 | 3.9 | .7 | 100 | 139 | .002 | 1.0 | 500 |
| WeJo | BMC | .5 | 100 | 15 | .8 | 100 | 17.4 | .003 | 1 | 333 |
| SLKA | BMC | 4 | 100 | 2.3 | .9 | 100 | 20.5 | .002 | 1 | 500 |
| TEKI | BMC | 5.5 | 100 | 1.2 | | 100 | 29 | .01 | 1 | 100 |
| MDR769¹ | BMC | 3 | 100 | 7.7 | .7 | 100 | 147 | .4 | 100 | 71 |
| ADA | onocytes | 100 | 100 | — | .7 | 100 | 10.3 | .007 | 1 | 131 |
| | | | | | | | | .5¹ | 100 | 18 |
| Ba-L | onocytes | 100 | 100 | — | .2 | 100 | 16 | .004 | 1 | 254 |
| | | | | | | | | .5¹ | 100 | 65 |
| SIVmac251 | BMC | 9 | 100 | 2 | .6 | 100 | 156 | .002 | 1 | 500 |
| HIV-2 (CDC | BMC | .1 | 100 | 19.5 | .7 | 100 | 60 | .0004 | 1 | 2439 |
| 310319) | | | | | | | | | | |

¹Dextran Sulfate (μg/ml) was used as a control compound.

Table 8 shows that DES 12.5% Sulfation was a more potent inhibitor of HIV-1 replication than DES 9.5% Sulfation. The IC₅₀, for DES 9.5% Sulfation ranged from 5.1 to >100 μg/ml (19-fold) and for DES 12.5% Sulfation from 0.6 to 11.7 μg/ml (19-fold range), thus their range of potencies against the v irus panel were equivalent. Although there is a basal difference in activity between the two compounds both compounds in general were active against the broad range of HIV-1 isolates tested, as well as displaying activity against a multi-drug resistant virus, SIV and HIV-2. Thus, these compounds are broadly anti-retroviral.

DES 12.5% Sulfation was active against all viruses tested. It was least active against the subtype C (IC₅₀ 10.3 μg/ml) and G (IC₅₀ 11.7 μg/ml) viruses. It also efficiently inhibited the replication of HIV-1 ADA. DES 12.5% Sulfation also displayed good antiviral activity with a clinical isolate of HIV-2 and the SIVmac251 isolate of SIV. It also displayed significant activity against the multi-drug resistant virus isolate MDR769. Thus, DES 12.5% Sulfation is active against a broad range of HIV-1 clinical isolates, multi-drug resistant viruses and other retroviruses.

DES 9.5% Sulfation showed a heterogeneous response (variation in IC₅₀) to the various viruses tested with activity ranging from inactive to active. DES 9.5% Sulfation has been previously demonstrated to be less active than DES 12.5% Sulfation in HIV-1 RoJo infected PBMCs, and this difference was again demonstrated here (37-fold less active). Examination of the antiviral curves for those viruses (ADA and Ba-L and the subtype G virus) for which DES 9.5% Sulfation was inactive suggests that it would be active at higher test concentrations. DES 9.5% Sulfation was also active against the MDR769 HIV-1 strain (IC₅₀ 13 μg/ml) and a clinical isolate of HIV-2 (IC₅₀ 5.1 μg/ml). Thus, despite lower over all potency it is still highly potent against multi-drug resistant HIV-1 and HIV-2.

DES 9.5% Sulfation and DES 12.5% Sulfation were tested against a range of HIV-1 clinical isolated and two other retroviruses (HIV-2 and SIV-1) and found to be broadly anti-retroviral. Additionally, these results show that the compounds are active against a resistant clinical isolate carrying the T215Y mutation for multi-drug resistance to RT inhibitors. The data also demonstrated that although DES 12.5% Sulfation was more potent than DES 9.5% Sulfation on an IC₅₀ basis, their range of IC₅₀s on the panel of viruses were comparable. Thus, it is likely that both inhibit virus replication via a comparable mechanism of action. Finally, the demonstration that the compounds are active against HIV-2 and SIV-1 show that they are applicable to other retroviruses.

6.8 Biodistribution of a Compound of the Invention

Male Sprague-Dawley rats obtained from Charles River Laboratories (Raleigh, N.C.; ca. 377-402 g) were dosed with [³H]Des6 40K by intravenous blus or oral gavage administration. Distribution of total tritium content in plasma, lymph, and cervical lymph nodes was quantitated in samples collected at 6 or 12 hours following dosing.

The study design is outlined in Table 9. Rats were divided into three treatment administration groups. Doses were formulated in phosphate buffered saline vehicle (pH=7.4) so as to deliver them in approximate volumes of 1.8 mL/kg (iv) and 2.1 mL/kg (oral gavage).

Prior to the time of biological sample collection at 6 or 12 hours after dosing, animals were anesthetized with ketamine/xylazine (7:1, ca. 120 mg/kg), and the thoracic lymphatic duct was cannulated as described in Waynforth, H. B. and Flecknell, Pa. (1992). Experimental and Surgical Technique in the Rat, 2nd ed., Academic Press, New York. At the time of sample analysis, blood was collected by cardiac puncture and lymph was collected via the lymphatic duct cannula. Blood was processed for plasma by centrifugation at ca. 1000 g for 10 minutes. Cervical lymph nodes were collected from each animal at times specified in Table 9. Except where noted, total radioactivity was quantitated in duplicate by liquid scintillation spectrometry for all biological samples collected.

TABLE 9
Study Design and Dosing

| Treatment Administration | | | | | | | | | |
|---|---|---|---|---|---|---|---|---|---|
| | | | Body | | Intended | | Actual | | Samples |
| | Rat | [³H]Des 6 40K | Weight | | Dose | | Dose | | Collection |
| Group | ID No. | Vial No. | kg | Route | mg/kg | μCi/kg | mg/kg | μCi/kg | Times h |
| 1 | G956 | B | 0.397 | IV | 28 | 189 | 27.8 | 204 | 6 |
| | G964 | B | 0.394 | | | | 27.4 | 201 | |
| | G965 | ND | 0.396 | | | | | | |
| 2 | G962 | A | 0.377 | IV | 28 | 189 | 29.2 | 209 | 12 |
| | G966 | A | 0.380 | | | | 30.0 | 210 | |
| | G961 | A | 0.376 | | | | 29.4 | 206 | |
| 3 | G960 | A | 0.392 | Oral | 33 | 223 | 34.9 | 245 | 12 |
| | G963 | B | 0.378 | | | | 31.9 | 234 | |
| | G958 | B | 0.402 | | | | 31.8 | 233 | |

ND - Animal not dosed due to insufficient amount of test article.

Results of the study are described in Table 10. In addition to listing total radiolabel content in plasma, lymph and lymph nodes; the lymph:plasma and lymph node:plasma ratios are also provided for each animal. Overall, concentrations of [³H]Des6 40K associated total radioactivity were highest in animals treated by iv administration, compared with oral administration, with highest concentrations in plasma and lymph at 6 h compared with 12 h. Plasma and lymph [³H]Des6 40K-eq concentrations at 12 h were approximately 1-2% of those obtained at 6 h. However, the concentrations of total radioactivity in lymph nodes were similar between these two time points following iv administration. Animal G961 in Group 2, died following anesthesia and prior to cannulation of the thoracic duct for collection of lymph. Lymph could not be collected from this animal; however plasma and lymph nodes were harvested for quantitation of total radioactivity. Total radioactivity in these collected biological media were found to be significantly greater than the other two rats that survived throughout the surgery. Lymph nodes in this animal were observed to be larger than the other two animals in Group 2.

Following oral administration of [³H]Des6 40K, concentrations of plasma total radioactivity were comparable to those obtained at 12 h following iv administration. Mean total radioactivity in lymph following oral administration was approximately 63% of those obtained at 12 h after iv administration; while total radioactivity in lymph nodes was only approximately 0.4% of those obtained following iv administration.

The lymph:plasma ratios increased in rats between the 6- and 12-h time points following iv administration (compare 0.14 and 0.64 vs. 1.7 and 1.3 for the 6- and 12-h time points, respectively), as plasma total radioactivity significantly decreased. The lymph/plasma ratios following oral administration were approximately one, indicating equal distribution of total radioactivity in these two media at 12 h.

The lymph node:plasma ratios increased to a much greater extent at the 12-h time point compared with those obtained at 6 h. The increase over time was much greater than those obtained in lymph over the same time course. These data suggest that [³H]Des6 40K associated radioactivity distributes to a large degree into lymph nodes and that the rate of elimination from this tissue is slow.

In contrast, the distribution profile in lymph nodes was different following oral administration, where the lymph node:plasma were only approximately 0.50-0.82, and demonstrate less distribution of total radioactivity into lymph nodes by the oral route.

TABLE 10
Total Radiolabel Content in Biological Samples

| | | | | | | Lymph | | | Lymph Nodes | | |
|---|---|---|---|---|---|---|---|---|---|---|---|
| | | Collection | | Plasma | | | | Tissue/ | | | Tissue/ |
| Group | Route | Time h | Rat ID | dpm-eq/g | ng-eq/g | dpm-eq/g | ng-eq/g | plasma | dpm-eq/g | ng-eq/g | Plasma |
| 1^{a} | IV | 6 | G956 | 399150 | 24504 | 56359 | 3460 | 0.14 | 433881 | 26636 | 1.1 |
| | | | G964 | 289861 | 17795 | 184598 | 11333 | 0.64 | 660045 | 40521 | 2.3 |
| | | | G965 | ND | ND | ND | ND | ND | ND | ND | ND |
| 2 | IV | 12 | G962 | 3243 | 208 | 5514 | 354 | 1.7 | 380783 | 24420 | 117 |
| | | | G966 | 4053 | 260 | 5292 | 339 | 1.3 | 466489 | 29917 | 115 |
| | | | G961^{b} | 12208 | 783 | ND | ND | ND | 843626 | 54103 | 69 |
| 3 | Oral | 12 | G960 | 3918 | 251 | 3828 | 246 | 0.98 | 1946 | 125 | 0.50 |
| | | | G963 | 3507 | 215 | 3366 | 207 | 0.96 | 2871 | 176 | 0.82 |
| | | | G958^{c} | 3311 | 203 | 3000 | 184 | 0.91 | ND | ND | ND |

^{a}Only enough dose was available to dose two rats in Group 1.
^{b}Rat G961 died after receiving anesthesia. No lymph fluid was obtained; however, plasma and lymph nodes were collected.
^{c}Only enough lymph for analysis of one aliquot was obtained. Cervical lymph nodes in Rat G958 could not be found, and were not collected.
ND—Not Determined.

The foregoing has demonstrated the pertinent and important features of the present invention. One of skill in the art will be appreciate that numerous modifications and embodiments may be devised. Therefore, it is intended that the appended claims cover all such modifications and embodiments.

1-55. (canceled)

56. A single unit dosage form of a pharmaceutical composition for treatment of microbial infection wherein said single unit dosage form comprises a therapeutically effective amount of a sulfated polysaccharide having a percent of sulfur greater than 6% and less than 13%, and a pharmaceutically-acceptable diluent or carrier, wherein said single unit dosage form is suitable for administration to a human.

57. A single unit dosage form of a pharmaceutical composition for treatment of microbial infections wherein said single unit dosage form comprises a therapeutically effective amount of a sulfated dextran having a percent of sulfur greater than 6% and less than 13% and a molecular weight of greater than 25,000, and a pharmaceutically-acceptable diluent or carrier, wherein said single unit dosage form is suitable for administration to a human.

58-60. (canceled)

61. The single unit dosage form of claim 56 which is formulated for topical, parenteral, oral, buccal or mucosal delivery.

62. The single unit dosage form of claim 56 which is formulated for controlled or delayed release.

63. The single unit dosage form of claim 56, wherein said sulfated polysaccharide has a percent of sulfur greater than or equal to 9.5% and less than 12.5%.

64. The single unit dosage form of claim 56, wherein said sulfated polysaccharide has a percent of sulfur greater than 12% and less than 13%.

65. The single unit dosage form of claim 56, wherein said sulfated polysaccharide has a molecular weight of at least 70,000.

66. The single unit dosage form of claim 56 which comprises a second active ingredient.

67. The single unit dosage form of claim 56, wherein said sulfated polysaccharide is a co-charged anionic polysaccharide.

68. The single unit dosage form of claim 79, wherein said co-charged anionic polysaccharide is co-charged with carboxymethyl groups, sulfate groups, sulfonate groups, or a combination thereof.

69. The single unit dosage form of claim 80, wherein said sulfated polysaccharide is a carboxymethyl dextran having a sulfur content of about 9.5% and an average molecular weight of about 20,000.

70. The single unit dosage form of claim 57 which is formulated for topical, parenteral, oral, buccal or mucosal delivery.

71. The single unit dosage form of claim 57 which is formulated for controlled or delayed release.

72. The single unit dosage form of claim 57, wherein said sulfated polysaccharide has a percent of sulfur greater than or equal to 9.5% and less than 12.5%.

73. The single unit dosage form of claim 57, wherein said sulfated polysaccharide has a percent of sulfur greater than 12% and less than 13%.

74. The single unit dosage form of claim 57, wherein said sulfated polysaccharide has a molecular weight of at least 70,000.

75. The single unit dosage form of claim 57 which comprises a second active ingredient.

76. The single unit dosage form of claim 94 further comprising a second active ingredient, wherein said second active ingredient is a second sulfated polysaccharide different from said sulfated polysaccharide.

77. The single unit dosage form of claim 57, wherein said sulfated polysaccharide is a co-charged anionic polysaccharide.

78. The single unit dosage form of claim 99, wherein said co-charged anionic polysaccharide is co-charged with carboxymethyl groups, sulfate groups, sulfonate groups, or a combination thereof.
